(19) United States
(12) Patent Application Publication
(10) Pub. No.: US 2024/0342310 A1
(43) Pub. Date: Oct. 17, 2024

(54) CCDC7 CIRCULAR RNA ENCODED PROTEIN INHIBITS THE PROGRESSION OF PROSTATE CANCER BY UP-REGULATING FLRT3

(71) Applicants: University of Virginia Patent Foundation, Charlottesville, VA (US); Sun Yat Sen Memorial Hospital, Sun Yat Sen University, Guangzhou (CN)

(72) Inventors: Hui Li, Charlottesville, VA (US); Qiong Wang, Guangzhou (CN); Hai Huang, Guangzhou (CN)

(21) Appl. No.: 18/625,283

(22) Filed: Apr. 3, 2024

Related U.S. Application Data
(60) Provisional application No. 63/496,212, filed on Apr. 14, 2023.

(51) Int. Cl. A61K 48/00; A61K 38/00; A61P 35/00; C07K 14/47

(52) U.S. Cl. CPC A61K 48/005; A61P 35/00; C07K 14/47; A61K 38/00; C07K 2319/02

(57) ABSTRACT

Disclosed herein are peptides and peptidomimetics that inhibit the progression of prostate cancer by up-regulating FLRT3. Also disclosed are methods for treating an FLRT3-attenuated cancer in a subject that involve administering to the subject the disclosed polypeptide or peptidomimetic or a polynucleotide encoding the disclosed peptide.

### CROSS-REFERENCE TO RELATED APPLICATIONS

This application claims benefit of U.S. Provisional Application No. 63/496,212, filed Apr. 14, 2023, which is hereby incorporated herein by reference in its entirety.

### SEQUENCE LISTING

This application contains a sequence listing filed in ST.26 format entitled “222117-1160 Sequence Listing” created on Mar. 28, 2024, and having 35,454 bytes. The content of the sequence listing is incorporated herein in its entirety.

### BACKGROUND OF THE INVENTION

Prostate cancer (PCa) is the most common cancer diagnosed in men in the USA, with 191,930 new diagnosed cases in 2020 which account for 21% of all cases in men (Siegel R L, et al. CA Cancer J Clin. 2020 70(1): 7-30). Compared with other tumors, PCa is often multifocal, having topographically and morphologically distinct tumor foci due to its special anatomical structure (Haffner M C, et al. Nat Rev Urol. 2021 18(2): 79-92). Different tumor foci within the same patients were reported to be genetically distinct, only rarely sharing any common somatic gene mutations, including those in cancer driver genes (Løvf M, et al. Eur Urol. 2019 75(3): 498-505). The multifocal and heterogeneous nature of PCa are important contributors to the difficulties associated with PCa diagnosis and treatment. Gleason score has historically been the most important morphological assessment tool for localized PCa, because it includes morphological characteristics of multiple lesions (Sehn J K. Mo Med. 2018 115(2): 151-155). Unfortunately, only the highest Gleason score of a multiple lesion is used as a criterion in clinical diagnosis, leading to inaccurate assessment and error in PCa study.

### SUMMARY OF THE INVENTION

Disclosed herein are peptides and peptidomimetics that inhibit the progression of prostate cancer by up-regulating FLRT3. For example, disclosed herein is a synthetic linear polynucleotide with the nucleic acid sequence SEQ ID NO:2, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2, a fragment thereof encoding at least amino acids 174-180 of SEQ ID NO: 1, or a combination thereof, wherein the linear polynucleotide comprises a cap and poly (A) tail.

Also disclosed herein is a peptidomimetic having the amino acid sequence SEQ ID NO: 1, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:1, a fragment thereof having at least amino acids 174-180 of SEQ ID NO:1, or a combination thereof. In some embodiments, the peptidomimetic is a fragment having at least 10, 20, 30, 40, 50, 60, 70, 80, 90, 100, 110, 120, 130, 140, 150, 160, 170, 180 contiguous amino acids from SEQ ID NO:1.

In some embodiments, the peptidomimetic further has a signal peptide, a C-terminal tag, or a combination thereof.

In some embodiments the peptidomimetic has the amino acid sequence

(SEQ ID NO: 3)
KMTEEQTYQAAEKSQESSTSGN,
(SEQ ID NO: 4)
MTEEQTYQAAEKSQESSTSGN,
(SEQ ID NO: 5)
TEEQTYQAAEKSQESSTSGN,
(SEQ ID NO: 6)
EEQTYQAAEKSQESSTSGN,
(SEQ ID NO: 7)
EQTYQAAEKSQESSTSGN,
(SEQ ID NO: 8)
QTYQAAEKSQESSTSGN,
(SEQ ID NO: 9)
TYQAAEKSQESSTSGN,
(SEQ ID NO: 10)
YQAAEKSQESSTSGN,
(SEQ ID NO: 11)
QAAEKSQESSTSGN,
(SEQ ID NO: 12)
AAEKSQESSTSGN,
(SEQ ID NO: 13)
AEKSQESSTSGN,
(SEQ ID NO: 14)
EKSQESSTSGN,
(SEQ ID NO: 15)
KSQESSTSGN,
(SEQ ID NO: 16)
SQESSTSGN,
(SEQ ID NO: 17)
QESSTSGN,
or
(SEQ ID NO: 18)
ESSTSGN.

Also disclosed herein is a chimeric polypeptide having the amino acid sequence SEQ ID NO:1, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:1, a fragment thereof having at least amino acids 174-180 of SEQ ID NO:1, or a combination thereof, fused to a signal peptide. In some embodiments, the chimeric polypeptide further includes a C-terminal tag.

In some embodiments the fragment of SEQ ID NO:1 has at least 10, 20, 30, 40, 50, 60, 70, 80, 90, 100, 110, 120, 130, 140, 150, 160, 170, 180 contiguous amino acids from SEQ ID NO:1. For example, in some embodiments, the fragment of SEQ ID NO:1 has the amino acid sequence KMTEEQTYQAAEKSQESSTSGN (SEQ ID NO:3), MTEEQTYQAAEKSQESSTSGN (SEQ ID NO:4), TEEQTYQAAEKSQESSTSGN (SEQ ID NO: 5), EEQTYQAAEKSQESSTSGN (SEQ ID NO:6), EQTYQAAEKSQESSTSGN (SEQ ID NO: 7), QTYQAAEKSQESSTSGN (SEQ ID NO:8), TYQAAEKSQESSTSGN (SEQ ID NO:9), YQAAEKSQESSTSGN (SEQ ID NO:10), QAAEKSQESSTSGN (SEQ ID NO:11), AAEKSQESSTSGN (SEQ ID NO:12), AEKSQESSTSGN (SEQ ID NO:13), EKSQESSTSGN (SEQ ID NO:14), KSQESSTSGN (SEQ ID NO:15), SQESSTSGN (SEQ ID NO:16), QESSTSGN (SEQ ID NO:17), or ESSTSGN (SEQ ID NO:18).

Also disclosed is a method for treating an FLRT3-attenuated cancer in a subject, the method involving administering to the subject a composition involving: (a)(i) a DNA expression vector expressing an mRNA polynucleotide comprising the nucleic acid sequence SEQ ID NO:2, or a variant thereof having at least 95% sequence identity to SEQ ID NO:2, a fragment thereof encoding at least amino acids 174-180 of SEQ ID NO:2; or a combination thereof, wherein the linear polynucleotide comprises a cap and poly (A) tail, or (a)(ii) a synthetic oligonucleotide comprising the nucleic acid sequence SEQ ID NO:2, or a variant thereof having at least 95% sequence identity to SEQ ID NO:2, a fragment thereof encoding at least amino acids 174-180 of SEQ ID NO:2; or a combination thereof, wherein the linear polynucleotide comprises a cap and poly (A) tail, or (a)(iii) a polypeptide or peptidomimetic having the amino acid sequence SEQ ID NO:1, or a variant thereof having at least 95% sequence identity to SEQ ID NO: 1, a fragment thereof having at least amino acids 174-180 of SEQ ID NO:1, or a combination thereof; and (b) a pharmaceutically acceptable excipient.

In some embodiments the FLRT3-attenuated cancer is a prostate cancer, pancreatic cancer, lung cancer, renal clear cell cancer, or colon cancer.

In some embodiments the polypeptide or peptidomimetic is a fragment having at least 10, 20, 30, 40, 50, 60, 70, 80, 90, 100, 110, 120, 130, 140, 150, 160, 170, 180 contiguous amino acids from SEQ ID NO:1.

In some embodiments, the synthetic oligonucleotide has one or modified nucleosides, at least one modified internucleoside linkage, or a combination thereof. For example, in some embodiments the one or more modified nucleosides is a 2′ sugar modified nucleoside. As disclosed herein, the one or more 2′ sugar modified nucleoside can independently be selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. In some embodiments, the internucleoside linkages are phosphorothioate internucleoside linkages.

Also disclosed is a method for treating a FLRT3-attenuated cancer in a subject, the method involving (a) determining the level of circCCDC7₁₉₋₁₅ and/or circCCDC7-180aa in said FLRT3-attenuated cancer; (b) comparing detected level(s) to level(s) in non-pancreatic cancer or level(s) in low Gleason score pancreatic tumors; (c) detecting a decrease in circCCDC7₁₉₋₁₅ and/or circCCDC7-180aa in said FLRT3-attenuated cancer as compared to non-pancreatic cancer levels or low Gleason score pancreatic tumor levels; and (d) treating the subject with FLRT3 or a vector encoding FLRT3.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1H show the discovery of circCCDC7(15, 16, 17, 18, 19) in PCa. FIG. 1A shows the pipeline for discovering prostate cancer chimeric RNAs: 1, based on MR reports, we focused on patients having tumor sites on two separate lobes; 2, during the surgery, the target tumors are dissected according to MR images; 3, half of the tumor was submitted to pathologist to receive a Gleason score evaluation for each tumor site; 4, selected tumors from the same patients with one lobe's Gleason score higher than 4+3, and another lower than 3+4; 5, six tumor tissues from three patients were obtained and submitted for RNA-Sequencing. Finally, EricScript software was performed to predict chimeric RNAs. FIG. 1B shows a gel image of RT-qPCR product of CCDC7₁₉₋₁₅ and CCDC7₁₉₋₁₃ in three clinical patients. Mix cDNA from tumor and adjacent normal margin was used. FIG. 1C shows Sanger sequencing results of the validated CCDC7₁₉₋₁₅ and CCDC7₁₉₋₁₃. FIG. 1D shows the schematic diagram of CCDC7₁₉₋₁₅ and CCDC7₁₉₋₁₃. FIG. 1E shows confirmation for CCDC7₁₉₋₁₅ and CCDC7₁₉₋₁₃ both being circular RNAs which circFBXW7 was used as a control. Total mix RNA from 23 pairs PCa patients was treated with or without RNase R before performing RT-PCR. FIG. 1F shows the structures of two reported circular RNAs which share the same junction sequences with CCDC7₁₉₋₁₅ and CCDC7₁₉₋₁₃. FIG. 1G shows a gel image of Touch-down PCR products of CCDC7₁₉₋₁₅ using divergent primers. The complementary DNA was synthesized with random hexamer primer using total mix RNA that from 23 pairs PCa patients treated with or without RNase R. FIG. 1H shows the schematic diagram of different isoforms of cirCCDC7.

FIGS. 2A to 2I show circCCDC7(15, 16, 17, 18, 19) was downregulated in tumor samples and associated with good prognosis. FIGS. 2A to 2B show expression of circCCDC7(15, 16, 17, 18, 19) (FIG. 2A) and linear CCDC7 (FIG. 2B) in 134 pairs of PCa and normal margin samples from CPGEA by using Mann-Whitney test. FIG. 2C shows the absence of correlation between circCCDC7(15, 16, 17, 18, 19) and linear CCDC7 in CPGEA. FIG. 2D to 2E shows quantitative PCR measuring the expression of circCCDC7(15, 16, 17, 18, 19) (FIG. 2D) and linear CCDC7 (FIG. 2E) in 23 pairs of clinical samples from Sun Yat-sen Memorial Hospital. The primers for linear CCDC7 were designed to amplify the junction sequences between exon40 and exon41 to avoid all reported circular transcripts. Mann-Whitney test was used to explore the group difference. FIG. 2F shows the absence of correlation between circCCDC7(15, 16, 17, 18, 19) and linear CCDC7 in Sun Yat-sen Memorial Hospital clinical samples. FIG. 2G shows expression of circCCDC7(15, 16, 17, 18, 19) in different Gleason scored samples from CPGEA. Mann-Whitney test was used to explore the group difference. FIG. 2H to 2I show BCR-free survival analysis of circCCDC7(15, 16, 17, 18, 19) (FIG. 2H) and linear CCDC7 (FIG. 2I) in CPGEA by using normalized read counts. *p<0.05. ****p<0.0001.

FIGS. 3A to 3G show circCCDC7(15, 16, 17, 18, 19) inhibits the viability, migration, and invasion of PCa. FIG. 3A shows colony formation assay measuring cell viability in DU145 and PC3 cell lines (left) and histogram quantification (right). FIG. 3B shows representative in vivo subcutaneous tumorigenesis images of the tumors of control group and circCCDC7(15, 16, 17, 18, 19) overexpression group using DU145. FIGS. 3C to 3D show tumor weight and volume were measured after the tumors were surgically dissected. FIGS. 3E to 3F, left panel, shows representative images of migration and invasion assays using DU145 (FIG. 3E) and PC3 (FIG. 3F) cells with or without circCCDC7(15, 16, 17, 18, 19) overexpression. FIGS. 3E to 3F, right panel, shows Histogram analysis of migrated and invaded cell counts. FIG. 3G shows representative image of the western blotting analysis of Slug, E-cad, and Claudin after circCCDC7(15, 16, 17, 18, 19) overexpression in DU145 and PC3 cells. The grayscale ratio of target protein to GAPDH was listed on top of the lanes. *p<0.05, **p<0.01, ***p<0.001.

FIGS. 4A to 4I show circCCDC7(15, 16, 17, 18, 19) encodes a protein. FIG. 4A shows the putative open reading frame (ORF) in circCCDC7(15, 16, 17, 18, 19). Note that the circCCDC7(15, 16, 17, 18, 19) junction is inside the ORF. FIG. 4B shows the comparison of ID index score of internal ribosomal entrance site (IRES) among circCCDC7(15, 16, 17, 18, 19), circFBXW7 and circZNF609 using IRESfinder. FIG. 4C shows the relative abundance of circFBXW7, circITCH, circZNF609, circCCDC7(15, 16, 17, 18, 19) and GAPDH mRNA in PC3 cell lines detected by RT-qPCR after actinomycin D treatment at the indicated time points. The relative expression was normalized to the expression of 0 h. FIG. 4D shows quantitative analysis of the expression of circCCDC7(15, 16, 17, 18, 19) between nuclear and cytoplasm in PC3 cells. FIG. 4E shows gel image of RT-qPCR product of the circFBXW7, circCCDC7(15, 16, 17, 18, 19), circITCH, and GAPDH in ribosome enriched RNA. Mixed ribosome RNA from LNCaP, C4-2, PC3 and DU145 were used. FIG. 4F shows FLAG tag antibody used to detect circCCDC7-180aa expression in 293T cells transfected with vector or overexpression plasmid. FIG. 4G shows circCCDC7-180aa was confirmed by immunoblotting after IP. The circCCDC7-180aa were extracted and subjected to mass spectrometry and junction-specific peptide (SQESSTSGN, SEQ ID NO:19) was identified (right panel). FIG. 4H shows immunofluorescence assay demonstrated that circCCDC7-180aa was mainly located around nucleus in HCT116 cell line. GFP plasmid and FLAG tagged circCCDC7(15, 16, 17, 18, 19) or empty vector were co-transfected. Upper is GFP+vector; Lower GFP+FLAG tagged circCCDC7(15, 16, 17, 18, 19). FIG. 4I shows a Western blot that showed circCCDC7-180aa specifically enriched in membrane fraction. FIG. 4J shows an ELISA assay that demonstrated circCCDC7-180aa is secreted into cell culture medium two days after plasmid transfection in 293T cells. ***p<0.001, ****p<0.0001.

FIGS. 5A to 5J shows circCCDC7(15, 16, 17, 18, 19) suppresses the progression of PCa by its encoded protein. FIG. 5A is a schematic diagram of circCCDC7(15, 16, 17, 18, 19) frame shift mutant which one base after the start codon (ATG) was deleted. FIG. 5B is a schematic diagram of linearized circCCDC7(15, 16, 17, 18, 19) ORF plasmid. FIG. 5C is a gel image of RT-PCR product of circCCDC7(15, 16, 17, 18, 19) from 293T cells transfected with vector, circCCDC7(15, 16, 17, 18, 19) overexpression or frame shift mutation group. FIG. 5D shows a Western blot demonstrated the absence of FLAG tagged protein with the frame shift mutant. FIG. 5E shows a colony formation assay measuring cell viability in DU145 cells when different plasmids were transfected. FIGS. 5F to 5G contains representative images of migration and invasion assays using PC3 after different plasmids transfection. FIGS. 5H to 5I show histogram analysis of migrated and invaded cell counts. FIG. 5J contains a representative image of the western blotting analysis of Slug, E-cad, and Claudin protein levels after transfection with different plasmids in PC3 cells. The grayscale ratio of target protein to GAPDH was listed on top of the lanes. ***p<0.001.

FIGS. 6A to 6D show circCCDC7(15, 16, 17, 18, 19) encodes a secretory protein that suppresses PCa. FIG. 6A shows colony formation assay measuring cell viability in PC3 and DU145 cells after supernatant from vector or circCCDC7(15, 16, 17, 18, 19) overexpression plasmid transfected 293T treatment. Supernatant to media ration, 1:1. FIGS. 6B to 6C contain representative images of migration and invasion assays of DU145 (FIG. 6B) and PC3 (FIG. 6C) with vector or oe supernatant treatment. Supernatant to media ration, 1:1. FIG. 6D contains representative images of immunofluorescence of PC3 with enriched supernatant treatment which from vector or circCCDC7(15, 16, 17, 18, 19) overexpression plasmid transfected 293T. *p<0.05, **p<0.01, **p<0.001.

FIGS. 7A to 7J show FLRT3 is a potential downstream of circCCDC7-180aa. FIGS. 7A to 7B show gene ontology analyses of differentially expressed genes after circCCDC7(15, 16, 17, 18, 19) overexpression in PC3 (FIG. 7A) and DU145 (FIG. 7B) cell lines. FIG. 7C shows three downstream genes selected from the RNA-seq were examined with TCGA database and TCGA relapse-free survival analyses. FIG. 7D shows relapse-free survival analysis of FLRT3 in TCGA. FIG. 7E shows expression of FLRT3 in 134 pairs of PCa and normal margin samples from CPGEA. FIGS. 7F and 7G show expression of FLRT3 in PCa and normal margin samples from TCGA (FIG. 7F) and GSE134051 (FIG. 7G). FIGS. 7H and 7I show expression of FLRT3 in normal, localized and metastasis samples from GSE6919 (FIG. 7H) and GSE3325 (FIG. 7I). FIG. 7J show expression of FLRT3 protein in PCa samples of different grades from Proteinatlas. *p<0.05, **p<0.01, ****p<0.0001.

FIGS. 8A to 8F show knocking down FLRT3 offsets the effect of circCCDC7(15, 16, 17, 18, 19) on PCa. FIG. 8A shows colony formation assay measuring cell viability with DU145 circCCDC7(15, 16, 17, 18, 19) overexpressing (oe) or control cells (vector) combined with FLRT3 knocking down (si1 and si2) or control siRNA (NC). FIGS. 8B and 8C contain representative images of migration and invasion assays using DU145 circCCDC7(15, 16, 17, 18, 19) oe combined with FLRT3 knocking down. FIGS. 8D to 8E show histogram analysis of migrated and invaded cell counts. FIG. 8F contains representative image of the western blotting analysis of Slug, E-cad, and Claudin protein levels using DU145 circCCDC7(15, 16, 17, 18, 19) oe combined with FLRT3 knocking down. The grayscale ratio of target protein to GAPDH was listed on top of the lanes. *p<0.05, **p<0.01, ***p<0.001.

FIG. 9 contains representative images of one of the PCa samples that met our selection criteria. The two tumor sites were distributed on two different lobes. Among those sites, left site: Gleason score=3+3, right site: Gleason score=4+4. In total, six samples were obtained from three patients.

FIG. 10 is a gel image of RT-qPCR product of CCDC7₁₉₋₁₅ and CCDC7₁₉₋₁₃ in another two clinical patients.

FIG. 11A to 11J show characterization of circCCDC7(15, 16, 17, 18, 19). FIG. 11A is a schematic diagram of circCCDC7(15, 16, 17, 18, 19) and specific primers design. FIG. 11B is a gel image of RT-PCR product of two primers for both forms and specific forms. FIGS. 11C-11D show comparison of the expression of circCCDC7(15, 16, 17, 18, 19) (FIG. 11C) and linear CCDC7 (FIG. 11D) in 134 pairs of PCa and normal margin samples from CPGEA using paired t test. FIG. 11E shows expression of linear CCDC7 from TCGA. FIG. 11F shows quantitative analysis of circCCDC7(15, 16, 17, 18, 19) in 23 pairs of clinical samples from Sun Yat-sen Memorial Hospital using paired t test. FIG. 11G shows expression of circCCDC7(15, 16, 17, 18, 19) in metastasis dataset GSE99857 after AGREP analysis. FIG. 11H shows relapse-free survival analysis of linear CCDC7 using FPKM value TCGA. FIGS. 11I-11J show AUC value of the ROC for normal or tumor by using the expression of circCCDC7(15, 16, 17, 18, 19) and linear CCDC7. *p<0.05, ****p<0.0001.

FIGS. 12A to 12F show the validation of PC3 and DU145 overexpression system. FIGS. 12A-12B show the change of ΔCq in PC3 and DU145 after circCCDC7(15, 16, 17, 18, 19) overexpressed. FIG. 12C is a gel image of RT-qPCR product of circCCDC7(15, 16, 17, 18, 19) when it was stable overexpressed in PC3 and DU145. FIG. 12D is a gel image of RT-qPCR product of circCCDC7(15, 16, 17, 18, 19) in prostate related cell lines. FIG. 12E shows the ΔCq value of circCCDC7(15, 16, 17, 18, 19) in prostate related cell lines. FIG. 12F shows the ΔCq value of circCCDC7(15, 16, 17, 18, 19) in 23 pairs of clinical samples from Sun Yat-sen memorial hospital. *p<0.05, ****p<0.0001.

FIGS. 13A to 13E show CircCCDC7(15, 16, 17, 18, 19) inhibits the viability, migration, and invasion of PCa. FIGS. 13A-13B are CCK8 assays measuring cell proliferation in DU145 and PC3 cell lines transfected with circCCDC7(15, 16, 17, 18, 19) overexpression plasmid or control vector. FIG. 13C shows tumor volume increase curves of the circCCDC7(15, 16, 17, 18, 19) overexpression and control groups are summarized in the line chart after subcutaneous injection. FIG. 13D is a gel image of RT-qPCR product of circCCDC7(15, 16, 17, 18, 19) when LNCap was transfected with circCCDC7(15, 16, 17, 18, 19) plasmid. FIG. 13E contains representative images of migration and invasion assays using LNCaP after different plasmids transfection, and the histogram analysis of migrated and invaded cell counts.

FIGS. 14A and 14B show quantitative analysis on the expression of circFBXW7 (FIG. 14A) and circZNF609 (FIG. 14B) between nuclear and cytoplasm in PC3 cells.

FIGS. 15A to 15E show CircCCDC7(15, 16, 17, 18, 19) encodes a protein which can be secreted. FIG. 15A shows FLAG tag antibody was used to detect circCCDC7-180aa expression in PC3 and DU145 cells after transfection. FIG. 15B is an immunofluorescence assay demonstrating that circCCDC7-180aa was mainly located around nucleus in DU145 cell line which circCCDC7(15, 16, 17, 18, 19) was stable overexpressed. FIG. 15C shows the supernatant from 293T cells was lyophilized, following by Western blot to measure the secreted protein. FIGS. 15D-15E show the supernatant from 293T cells was used to do IP and mass spectrometry, which identified the peptides of circCCDC7-180aa.

FIGS. 16A to 16F show CircCCDC7(15, 16, 17, 18, 19) suppresses the cell viability, migration, and invasion of DU145 by its encoded protein. FIG. 16A is a colony formation assay measuring cell viability in DU145 cells when different plasmids were transfected. FIGS. 15B-15C contain representative images of migration and invasion assays using DU145 after different plasmids transfection. FIGS. 15D-15E show histogram analysis of migrated and invaded cell counts. FIG. 15F is a representative image of the Western blotting analyses of Slug, E-cad, and Claudin protein levels after different plasmids transfection. ***p<0.001.

FIGS. 17A to 17I show FLRT3 is a potential downstream of circCCDC7(15, 16, 17, 18, 19). FIGS. 17A-17B show relapse-free survival analysis of PLXDC1 (FIG. 17A) and COL10A1 (FIG. 17B) in TCGA. FIGS. 17C-17D show quantitative analysis expression of FLRT3 in PC3 and DU145 when circCCDC7(15, 16, 17, 18, 19) was overexpressed.

FIG. 17E is a representative image of the Western blotting analysis of FLRT3 protein levels when circCCDC7(15, 16, 17, 18, 19) was overexpressed in DU145 and PC3. The grayscale ratio of FLRT3 to GAPDH was listed on top of the lanes. FIGS. 17F-17G show quantitative analysis expression of FLRT3 in PC3 and DU145 when treated with supernatant from circCCDC7(15, 16, 17, 18, 19) overexpressed 293T cells. FIG. 17H shows the correlation between circCCDC7(15, 16, 17, 18, 19) and FLRT3 in CPGEA dataset. FIG. 17| contains photomicrographs of the immunohistochemical staining of FLRT3 in the Proteinatlas. *p<0.05, ***p<0.001.

FIGS. 18A and 18B show RT-qPCR (FIG. 18A) and Western blot (FIG. 18B) analysis of FLRT3 expression levels in FLRT3-knockdown cells with circCCDC7(15, 16, 17, 18, 19) overexpressing combined with FLRT3 knocking down. The grayscale ratio of FLRT3 to GAPDH was listed on top of the lanes. **p<0.01.

FIG. 19 is a schematic diagram of circCCDC7(15, 16, 17, 18, 19) influencing prostate cancer.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules.

### Polynucleotides

In some embodiments, CCDC7₁₉₋₁₅ RNA has the nucleic acid sequence:

ATGCAAGTGGGTAATAGTCAAACAAAAGTTAAAGGTGAAGATTCAAAAAATATACCATTGGA
GAAAGAAACAAGAAAATCACTGGTTTCAGATTCAGGTGGACAAAGGACAAGTGATAAAATC
CAAGAATATCCACAGATCACTGCCCAAAGCGGAAGACTGATTGAAAAGAGCTCAGAGAAGA
AACGATCTAGCCCTGCTATTTCAGATCTTTCACAGATCCTTAAGTCTCAAGATGAATCAGCA
TTTTTAGAGAGTTCAAATGAAGTTTCAGTTGCTGAAAACCAATCCTATAAATCTCCATCAGA
GACCCATGATAAATCACTTACAACAGTATCATCCAGCAAAGAAGTTCAAGATTCACTGTCTG
TTGGAACATTGGCTCAAAAAAACGAAACAGTGATATCACCATTCATTTTACCTCCTGTTCTT
ACAGAAAGTAAAAAGGCTGATGTTTCAGAAGAACAATTACAAAAGATGACTGAAGAACAAA
CTTACCAAGCAGCAGAAAAATCTCAAG-AAAGTAGCACGTCTGGAAATTGA (SEQ ID NO: 2,
C-terminal addition from circularization bolded).

Therefore, disclosed herein is a synthetic linear polynucleotide with the nucleic acid sequence SEQ ID NO:2, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2, a fragment thereof encoding at least amino acids 174-180 of SEQ ID NO:1, or a combination thereof, wherein the linear polynucleotide comprises a 5′ capping region (5′ cap), a 3′ tailing sequence, e.g., a poly-A tail, or a combination thereof.

The synthetic polynucleotides of the invention can include a 5′ capping region or 5′ cap. The 5′ cap structure of an mRNA is involved in nuclear export, increasing mRNA stability and binds the mRNA Cap Binding Protein (CBP), which is responsible for mRNA stability in the cell and translation competency through the association of CBP with poly (A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to the polynucleotide may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which may equivalently be designated 3′ 0-Me-m7G (5′) ppp (5′) G). The 3′-O atom of the other, unmodified, guanine becomes linked to the 5′-terminal nucleotide of the capped nucleic acid molecule (e.g. an mRNA or mmRNA). The N7- and 3′-O-methlyated guanine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

While cap analogs allow for the concomitant capping of a nucleic acid molecule in an in vitro transcription reaction, up to 20% of transcripts remain uncapped. This, as well as the structural differences of a cap analog from an endogenous 5′-cap structures of nucleic acids produced by the endogenous, cellular transcription machinery, may lead to reduced translational competency and reduced cellular stability.

Polynucleotides disclosed herein may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the CapI structure. This cap results in a higher translational-competency and cellular stability and a reduced activation of cellular pro-inflammatory cytokines, as compared, e.g., to other 5′cap analog structures known in the art. Cap structures include 7 mG (5′) ppp (5′) N,pN2p (cap 0), 7 mG (5′) ppp (5′) NImpNp (cap 1), and 7 mG (5′)-ppp (5′) NImpN2mp (cap 2).

Therefore, the 5′ terminal caps may include endogenous caps or cap analogs. In some embodiments, the 5′ terminal cap may comprise a guanine analog. Useful guanine analogs include inosine, N1-methyl-guanosine, 2′fluoro-guanosine, 7-deaza-guanosine, 8-oxo-guanosine, 2-amino-guanosine, LNA-guanosine, and 2-azido-guanosine.

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between 100 and 250 residues long.

Generally, the length of a poly-A tail is greater than 30 nucleotides in length. In some embodiments, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide. This design may be based on the length of the coding region, the length of a particular feature or region (such as the first or flanking regions), or based on the length of the ultimate product expressed from the polynucleotides, primary constructs or mmRNA.

In this context the poly-A tail may be 10, 20, 30, 40, 50, 60, 70, 80, 90, or 100% greater in length than the polynucleotide. The poly-A tail may also be designed as a fraction of polynucleotide to which it belongs. In this context, the poly-A tail may be 10, 20, 30, 40, 50, 60, 70, 80, or 90% or more of the total length of the construct or the total length of the construct minus the poly-A tail.

### Peptides and Peptidomimetics

In some embodiments, CCDC7₁₉₋₁₅ RNA encodes the protein sequence:

MQVGNSQTKVKGEDSKNIPLEKETRKSLVSDSGGQRTSDKIQEYPQITAQSGRLIEKSSEKKRS
SPAISDLSQILKSQDESAFLESSNEVSVAENQSYKSPSETHDKSLTTVSSSKEVQDSLSVGTLA
QKNETVISPFILPPVLTESKKADVSEEQLQKMTEEQTYQAAEKSQESSTSGN (SEQ ID NO: 1,
C-terminal addition from circularization bolded).

Also disclosed herein is a peptidomimetic having the amino acid sequence SEQ ID NO: 1, or a variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:1, a fragment thereof having at least amino acids 174-180 of SEQ ID NO:1, or a combination thereof. In some embodiments, the peptidomimetic is a fragment having at least 10, 20, 30, 40, 50, 60, 70, 80, 90, 100, 110, 120, 130, 140, 150, 160, 170, 180 contiguous amino acids from SEQ ID NO:1.

In some embodiments, the peptidomimetic further has a signal peptide, a C-terminal tag, or a combination thereof.

In some embodiments the peptidomimetic has the amino acid sequence

(SEQ ID NO: 3)
KMTEEQTYQAAEKSQESSTSGN,
(SEQ ID NO: 4)
MTEEQTYQAAEKSQESSTSGN,
(SEQ ID NO: 5)
TEEQTYQAAEKSQESSTSGN,
(SEQ ID NO: 6)
EEQTYQAAEKSQESSTSGN,
(SEQ ID NO: 7)
EQTYQAAEKSQESSTSGN,
(SEQ ID NO: 8)
QTYQAAEKSQESSTSGN,
(SEQ ID NO: 9)
TYQAAEKSQESSTSGN,
(SEQ ID NO: 10)
YQAAEKSQESSTSGN,
(SEQ ID NO: 11)
QAAEKSQESSTSGN,
(SEQ ID NO: 12)
AAEKSQESSTSGN,
(SEQ ID NO: 13)
AEKSQESSTSGN,
(SEQ ID NO: 14)
EKSQESSTSGN,
(SEQ ID NO: 15)
KSQESSTSGN,
(SEQ ID NO: 16)
SQESSTSGN,
(SEQ ID NO: 17)
QESSTSGN,
or
(SEQ ID NO: 18)
ESSTSGN.

In some embodiments, the disclosed polypeptide is a peptidomimetic. As used herein, “peptidomimetic” means a mimetic of a peptide which includes some alteration of the normal peptide chemistry. Peptidomimetics typically enhance some property of the original peptide, such as increase stability, increased efficacy, enhanced delivery, increased half life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The disclosed composition can be linked to an internalization sequence or a protein transduction domain to effectively enter the cell. Recent studies have identified several cell penetrating peptides, including the TAT transactivation domain of the HIV virus, antennapedia, and transportan that can readily transport molecules and small peptides across the plasma membrane (Schwarze et al., Science. 1999 285(5433): 1569-72; Derossi et al. J Biol Chem. 1996 271(30): 18188-93; Yuan et al., Cancer Res. 2002 62(15): 4186-90). More recently, polyarginine has shown an even greater efficiency of transporting peptides and proteins across the plasma, membrane making it an attractive tool for peptide mediated transport (Fuchs and Raines, Biochemistry. 2004 43(9): 2438-44). Nonaarginine has been described as one of the most efficient polyarginine based protein transduction domains, with maximal uptake of significantly greater than TAT or antennapeadia. Peptide mediated cytotoxicity has also been shown to be less with polyarginine-based internalization sequences. R₉ mediated membrane transport is facilitated through heparan sulfate proteoglycan binding and endocytic packaging. Once internalized, heparan is degraded by heparanases, releasing R₉ which leaks into the cytoplasm (Deshayes et al., Cell Mol Life Sci. 2005 62(16): 1839-49). Studies have recently shown that derivatives of polyarginine can deliver a full length p53 protein to oral cancer cells, suppressing their growth and metastasis, defining polyarginine as a potent cell penetrating peptide (Takenobu et al., Mol Cancer Ther. 2002 1(12): 1043-9). Thus, the provided polypeptide can comprise a cellular internalization transporter or sequence. The cellular internalization sequence can be any internalization sequence known or newly discovered in the art, or conservative variants thereof. Non-limiting examples of cellular internalization transporters and sequences include Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol).

In some embodiments, the polypeptide is a chimeric molecule comprising a “targeting molecule” or “targeting moiety.” A targeting molecule is a molecule such as a ligand or an antibody that specifically binds to its corresponding target, for example a receptor on a cell surface. Thus, for example, where the targeting molecule is a ligand, the chimeric molecule will specifically bind (target) cells and tissues bearing expressing the receptor for that ligand.

### Pharmaceutical Compositions

Also disclosed is a composition comprising the disclosed polypeptide in a pharmaceutically acceptable excipient. Pharmaceutical carriers suitable for administration of the compounds provided herein include any such carriers known to those skilled in the art to be suitable for the particular mode of administration. The polypeptides can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the polypeptides described above are formulated into pharmaceutical compositions using techniques and procedures well known in the art (See, e.g., Ansel, Introduction to Pharmaceutical Dosage Forms, 4th Edition, 1985, 126).

In addition, the compounds may be formulated as the sole pharmaceutically active ingredient in the composition or may be combined with other active ingredients. For example, the compounds may be formulated or combined with known NSAIDs, anti-inflammatory compounds, steroids, and/or antibiotics.

In one embodiment, the compositions are formulated for single dosage administration. To formulate a composition, the polypeptide is dissolved, suspended, dispersed or otherwise mixed in a selected carrier at an effective concentration such that the treated condition is relieved or one or more symptoms are ameliorated.

In some embodiments, the peptide is formulated in a suitable peptide-delivery nanoparticle, such as encapsulated within nanoparticles of poly(lactide-co-glycolide) copolymer, cyclodextrin nanoparticles, or cetyl alcoholipolysorbate.

Peptides may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. For example, PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane.

To ensure full gastric resistance a coating can be impermeable to at least pH 5.0. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used. The peptides could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

To aid dissolution of peptides into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios.

In some embodiments, the polypeptide is encapsulated in a biocompatible nanoparticle. In particular embodiments, the polypeptide is formulated in a vehicle containing about 33% 2-Hydroxypropyl)-β-cyclodextrin (HPBCD) in PBS and about 45% polyethylene glycol 400.

### Methods of Administration and Treatment

The cancer of the disclosed methods can be any cell in a subject undergoing unregulated growth, invasion, or metastasis. In some aspects, the cancer is breast cancer, prostate cancer, or colon cancer. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed polypeptides can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed polypeptides can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016).

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

In one embodiment, the disclosed polypeptide compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of polypeptide administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

Although the polypeptide compositions may be administered once or several times a day, and the duration of the treatment may be once per day for a period of about 1, 2, 3, 4, 5, 6, 7 days or more, it can be more preferably to administer either a single dose in the form of an individual dosage unit or several smaller dosage units or by multiple administration of subdivided dosages at certain intervals. For instance, a dosage unit can be administered from about 0 hours to about 1 hr, about 1 hr to about 24 hr, about 1 to about 72 hours, about 1 to about 120 hours, or about 24 hours to at least about 120 hours. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours. Subsequent dosage units can be administered any time following the initial administration such that a therapeutic effect is achieved.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: A Protein-Encoding CCDC7 Circular RNA Inhibits the Progression of Prostate Cancer by Up-Regulating FLRT3

In this study, we utilized MRI imaging and pathological diagnosis to determine Gleason scores of two different tumor foci within the same patients, collected the samples, and conducted total RNA-Seq. Our initial goal was to uncover chimeric fusion RNAs that are differentially expressed in tumors with low vs. high Gleason scores. To our surprise, one transcript containing junction sequence from CCDC7 exon19 to exon15 of the same gene was found in only low Gleason (≤3+4) tissues. Subsequently, we confirmed that this is not a traditional intergenic chimeric RNA, but rather a circular RNA resulted from back splicing of exon19 to exon15 of CCDC7.

Circular RNAs are a family of endogenous RNAs which have become a focus of biological research in recent years (Zhou W Y, et al. Mol Cancer. 2020 19(1): 172). Studies have demonstrated that circular RNAs are involved in the progress of cell proliferation, apoptosis, metastasis, and therapy resistance in PCa (Huang C, et al. J Cell Mol Med. 2019 23(9): 6112-6119; Shen Z, et al. Cancer Lett. 2020 468:88-101; Chen Y, et al. Cell Death Differ. 2019 26(7): 1346-1364; Du S, et al. Cancer Manag Res. 2020 12:7487-7499; Liu X, et al. Mol Ther Nucleic Acids. 2021 26:1130-1147). They mainly play regulatory roles in the pathological process of PCa by acting as miRNA sponges or binding to proteins. Though a subset of circRNAs has been shown to be potentially translated into polypeptides (Granados-Riveron J T, et al. Biochim Biophys Acta. 2016 1859(10): 1245-51; Dhamija S, et al. RNA Biol. 2018 15(8): 1025-1031; Pamudurti N R, et al. Mol Cell. 2017 66(1): 9-21.e7), this kind of phenomenon has not been reported in PCa (Liu X, et al. Mol Ther Nucleic Acids. 2021 26:1130-1147).

Here we accumulated multiple lines of evidence supporting that circCCDC7(15, 16, 17, 18, 19) has tumor suppressive activities: 1) it is expressed at lower levels in high-Gleason tumors than low-Gleason ones; 2) it is also expressed at lower levels in prostate cancer than in matched normal margins; 3) overexpressing circCCDC7(15, 16, 17, 18, 19) resulted in the suppression of prostate cancer cell viability, migration, and invasion in vitro; 4) overexpressing circCCDC7(15, 16, 17, 18, 19) also yielded smaller tumors in vivo; and 5) low circCCDC7(15, 16, 17, 18, 19) expression is correlated with worse clinical outcome. Moreover, different from the more well-known mechanisms of function, circCCDC7(15, 16, 17, 18, 19) shared many common characteristics with protein-encoding circFBXW7 and circZNF606, and further experiments validated its protein translation activity, which is its mechanism of action for the phenotypes studied here. Additional studies found that circCCDC7(15, 16, 17, 18, 19) can inhibit the migration and invasion at least partially via FLRT3. Taken together, our study identifies a circular RNA in PCa, which may suppress the progress of PCa by encoding a protein at least partially through FLRT3.

### Results

### The Discovery of a Circular RNA in PCa

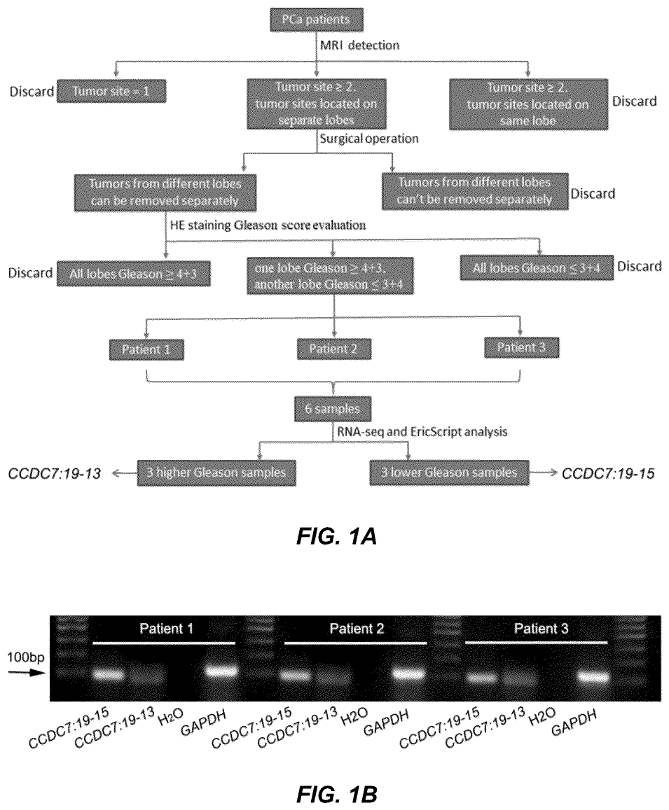

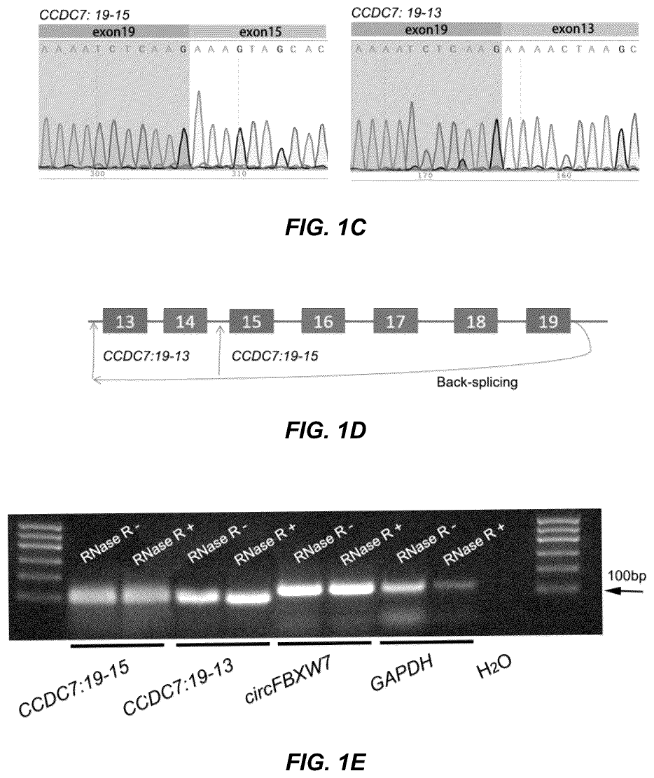

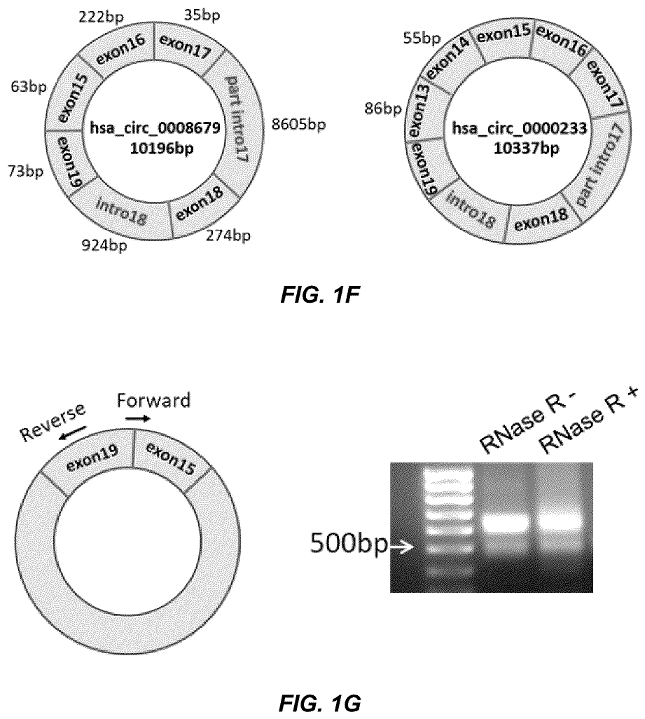

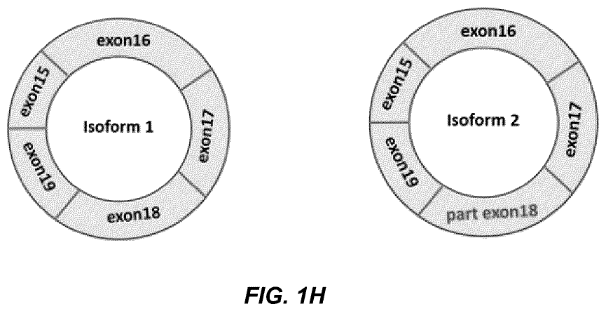

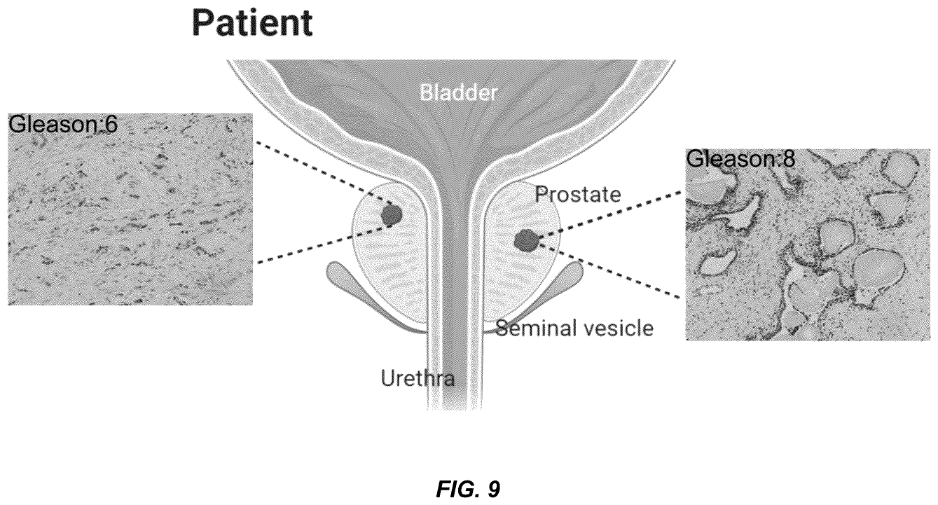

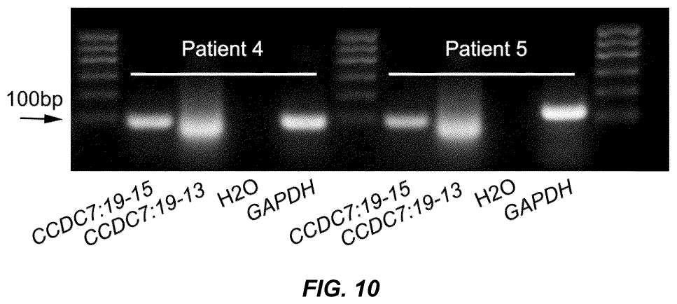

Specific gene fusions and their products (fusion RNA and protein) have been widely used as cancer diagnostic markers and therapeutic targets for many years (Li Z, et al. Curr Opin Genet Dev. 2018 48:36-43). However, few recurrent fusions were found in prostate cancer with the exception of ETS family-associated gene fusions (Wang Q, et al. Cell Biosci. 2022 12(1): 153; Li H, et al. Cancer Pathogenesis and Therapy 2023 01:216-219). Recent work by our group and others has demonstrated that intergenic splicing represents an epitranscriptomic mechanism for chimeric fusion RNAs and potentially fusion proteins (Yun J W, et al. Genome Biol. 2020 21(1): 166; Shi X, et al. Adv Clin Chem. 2021 100:1-35; Wu H, et al. Genes Dis. 2019 6(4): 385-390; Wang L, et al. Cancer Lett. 2021 31:501:1-1121). We thus hypothesized that chimeric RNAs may play important role in the malignancy of PCa and contribute to its heterogeneity. We first collected three pairs of differentially-scored PCa tissues from different lobes of the same patients according to the selection process described in the Methods. Further evaluation by three pathologists validated the Gleason scores of these six samples, which were subsequently submitted for RNA-seq (FIG. 9). We then used the EricScript (Singh S, et al. Methods Mol Biol. 2020 2079:3-12) pipeline to identify recurrent chimeric RNAs. Those that specifically expressed in high Gleason score (≥4+3) or low Gleason score (≤3+4) samples were selected (FIG. 1A). To our amazement, only one transcript, an isoform of CCDC7 with a junction site of exon19 spliced to exon15 was detected in all three low Gleason samples. In contrast, another isoform of CCDC7 (exon19 joining to exon13) was found only in three high Gleason samples, suggesting that fusion CCDC7₁₉₋₁₅ was a potential tumor suppressor gene and fusion CCDC7₁₉₋₁₃ may have opposite effect. We first designed primers flanking the fusion junction site (forward primers annealing to exon 19 and reverse to exon15 or 13) and used qPCR to amplify these two transcripts. Agarose electrophoresis revealed bright bands for both CCDC7₁₉₋₁₅ and CCDC7₁₉₋₁₃ (FIGS. 1B and 10A). Further Sanger sequencing validated the existence of these two transcripts in clinical samples (FIGS. 1C and 10B). We then tested whether CCDC7₁₉₋₁₅ and CCDC7₁₉₋₁₃ are both circular RNAs formed by back-splicing (FIG. 1D). We used RNase R treat the RNA before reverse transcription. the abundance of both forms did not decrease after treatment with RNase R, since GAPDH was clearly reduced, supporting that CCDC7₁₉₋₁₅ and CCDC7₁₉₋₁₃ are indeed circular RNAs (FIG. 1E). Interestingly, two circular RNAs with the same junction sequences are reported on circBank. hsa_circ_0008679(10196 bp) has the same junction to CCDC7₁₉₋₁₅, and hsa_circ_0000233(10337 bp) to CCDC7₁₉₋₁₃ (FIG. 1F). In this manuscript, we focused on CCDC7₁₉₋₁₅ for further study.

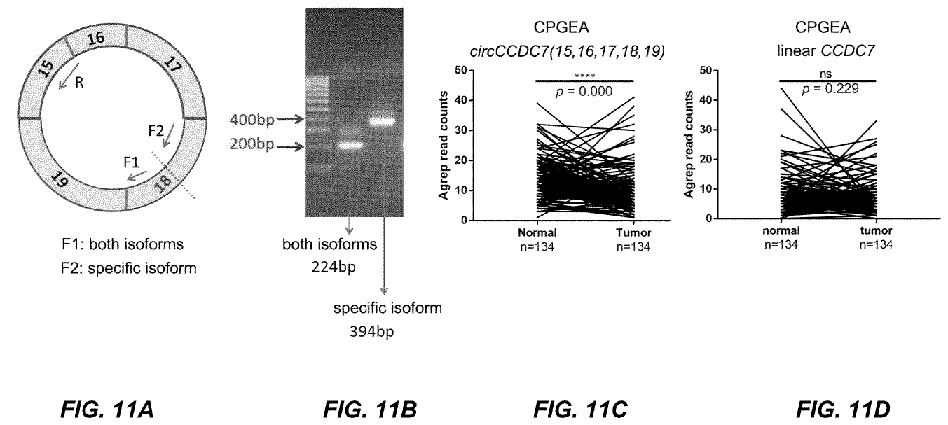

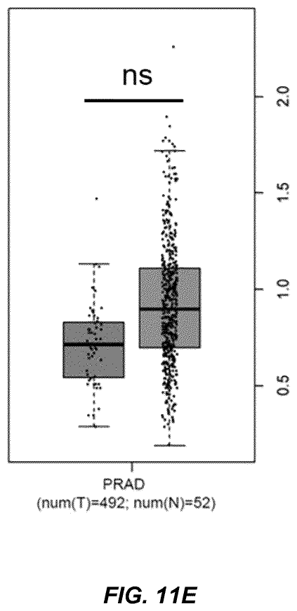

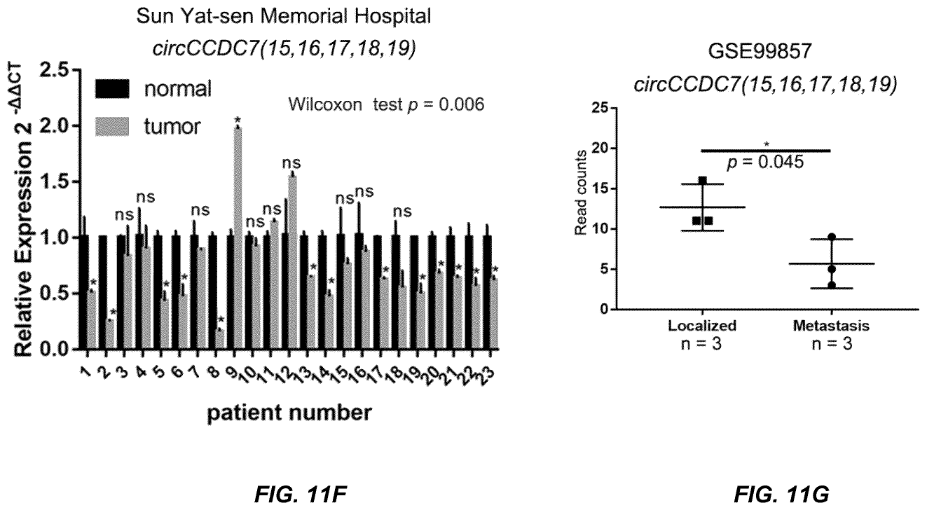

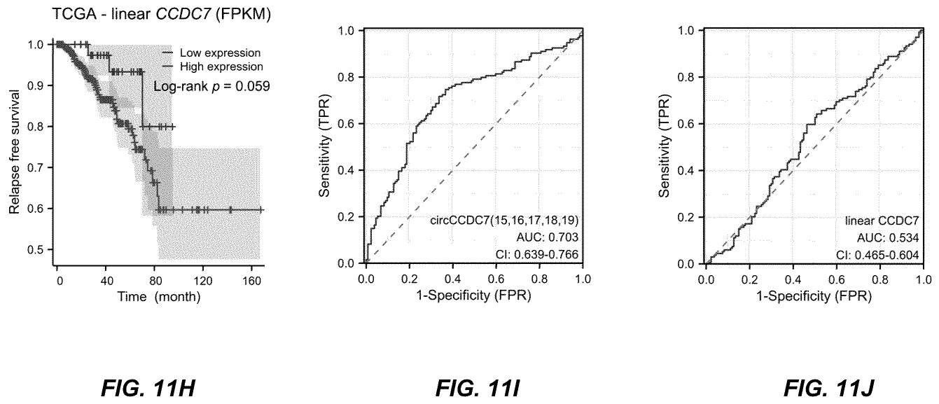

To test whether CCDC7₁₉₋₁₅ was the reported hsa_circ_0008679, we used divergent primer pairs in which the forward primer annealing to the exon19/15 junction sequence and the reverse primer located on exon19 (FIG. 1G). To avoid the amplification of the linear form, the full length of forward primer aligns to exon 19 and 15 was 22 bp, of which 9 bp from exon19 and 13 bp from exon15. Touch-down PCR with Platinum Taq High Fidelity Kit with capability to amplify 15 kb was used to amplify the full length of CCDC7₁₉₋₁₅. Interestingly, two bands between 500 bp and 700 bp were found (FIG. 1G), but not at the size of 10196 bp as in hsa_circ_0008679. Further Sanger sequencing demonstrated that the upper bright band is a circular RNA formed from exon15 to exon19 of CCDC7 (NM_001321115.2) with a full length of 667 bp (isoform1; FIGS. 1H and 10C). The slightly lower band is also formed from exon15 to exon19 of CCDC7, but part of exon18 is lost (isoform2; FIGS. 1H and 10D). We suspected a cryptic splicing site on exon18 leading to the formation of the isoform2 (FIG. 10D) (Murray J I, et al. Genome Biol. 2008 9(6): R97). According to the recent published guidelines of naming eukaryotic circular RNAs (Chen L L, et al. Nat Cell Biol. 2023 25(1): 1-5), we named the isoform1 as circCCDC7(15, 16, 17, 18, 19), and the isoform2 as circCCDC7(15, 16, 17, 18S, 19). Given that small products are favorably amplified by PCR, and the brightness of circCCDC7(15, 16, 17, 18S, 19) was much lower than that of circCCDC7(15, 16, 17, 18, 19), circCCDC7(15, 16, 17, 18S, 19) must be relatively lowly expressed. We therefore decided to focus on circCCDC7(15, 16, 17, 18, 19). To distinguish the isoforms, we designed another two forward primers as shown in FIG. 11A. F1 can amplify both isoforms, whereas F2 can specifically amplify isoform1. Further agarose electrophoresis and Sanger sequencing verified the specificity of F2 (FIG. 11B). F2 was subsequently employed for RT-qPCR in clinical samples to measure the expression level of circCCDC7(15, 16, 17, 18, 19).

CircCCDC7(15, 16, 17, 18, 19) is Downregulated in PCa and its Expression is Associated with Better Prognosis

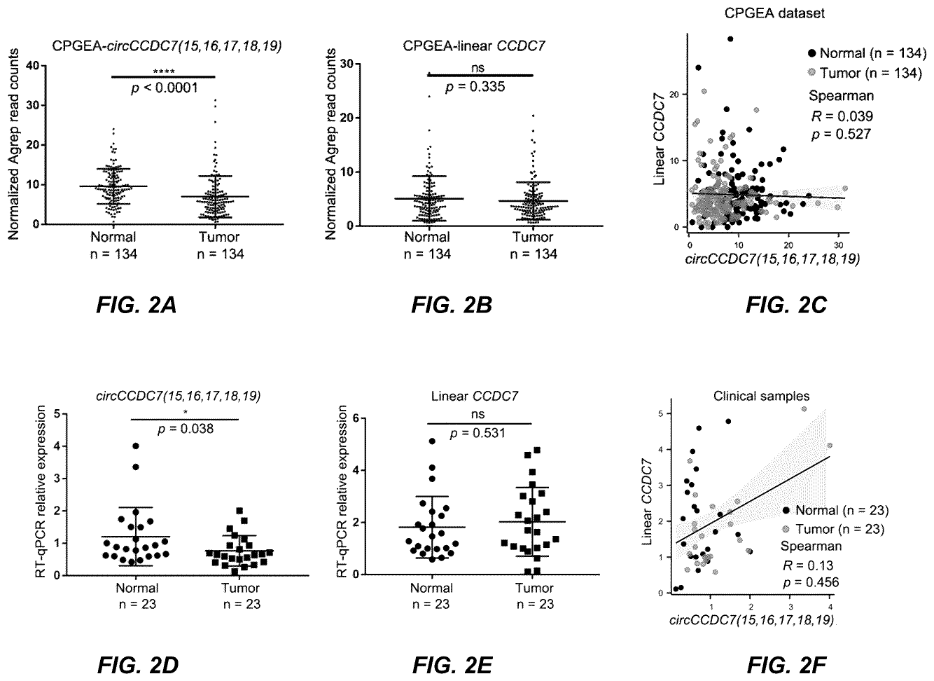

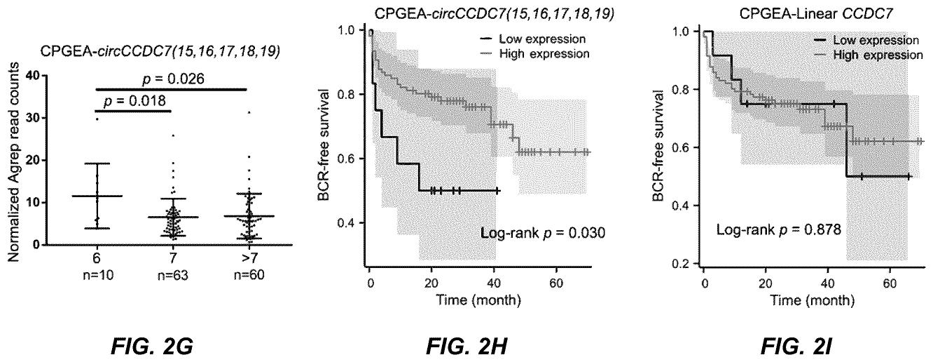

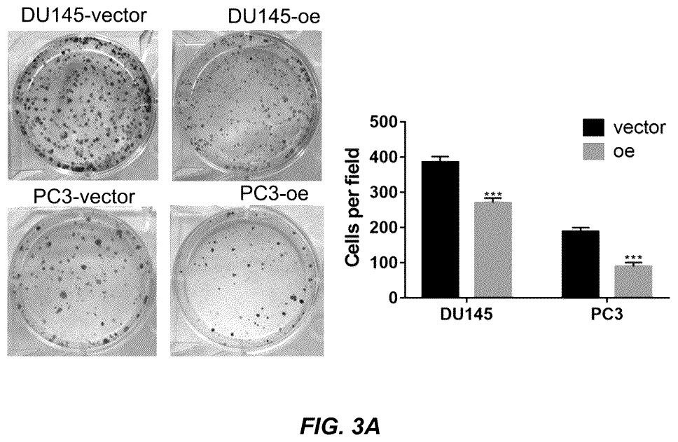

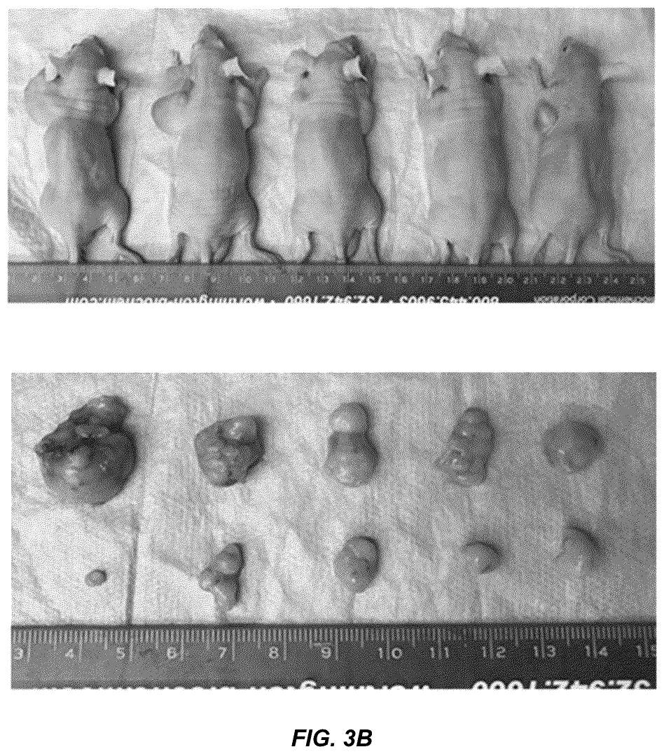

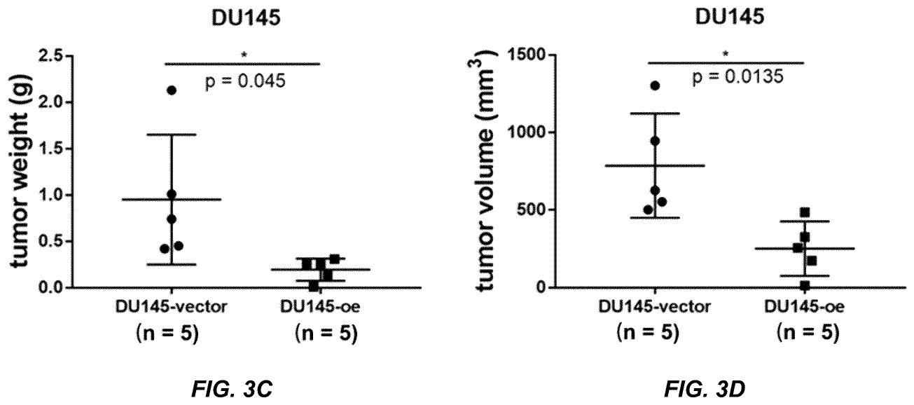

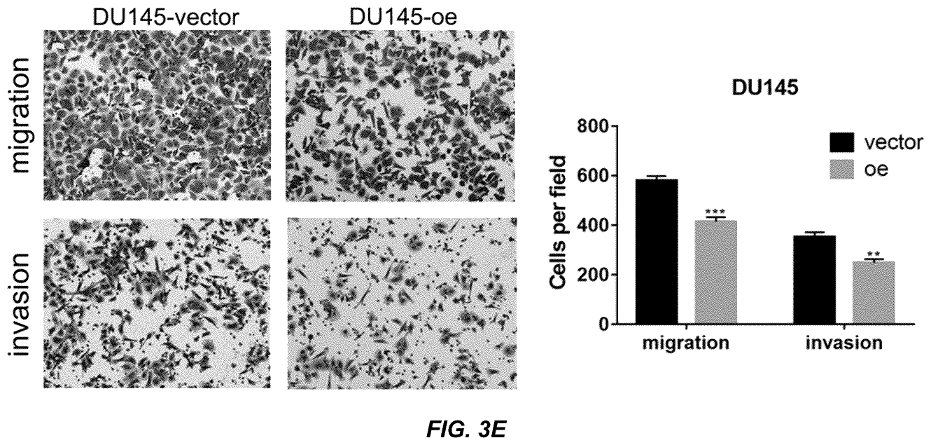

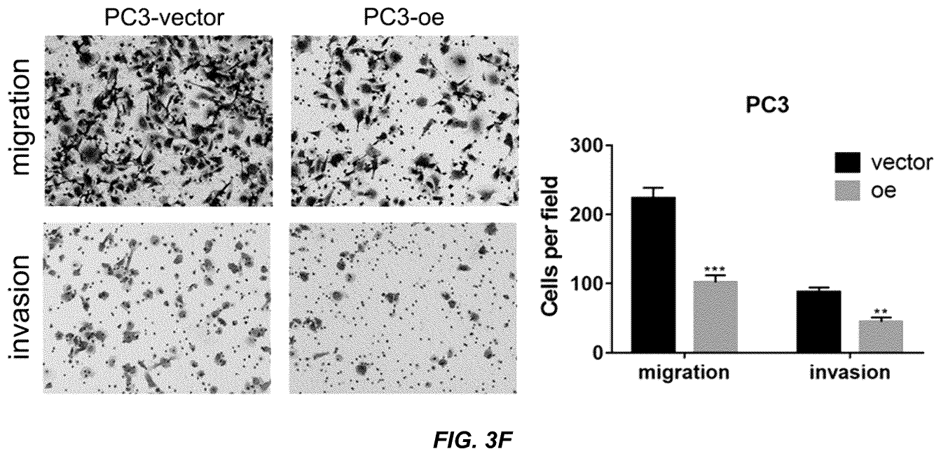

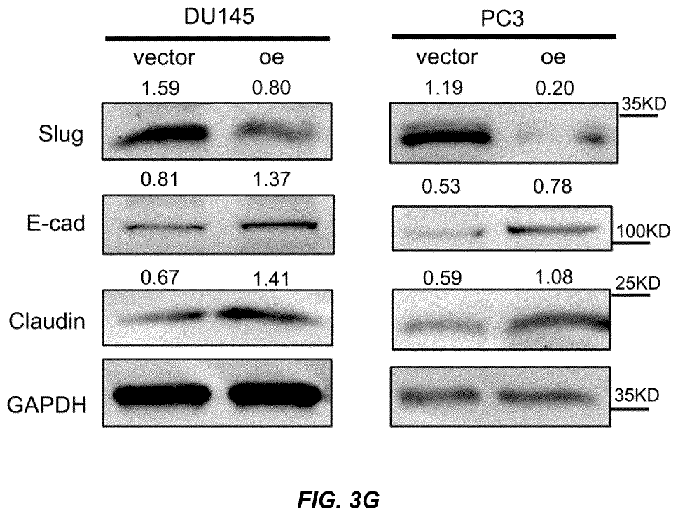

To investigate the association of circCCDC7(15, 16, 17, 18, 19) with PCa, AGREP analysis was performed to quantify its expression by searching for the junction sequence in CPGEA RNA-seq data. We first compared the expression difference between tumor and matched normal tissues and found that the circCCDC7(15, 16, 17, 18, 19) was expressed significantly lower in tumor samples than in the paired normal margins (FIGS. 2A and 11C). In contrast, when we used the AGREP to search for the junction sequence of CCDC7 exon 19 and 20 (representing the linear CCDC7), no statistical significance was observed between tumors and normals (FIGS. 2B and. 11D). The same situation was found in the TCGA database (Supplementary FIG. 3e). Consistently, we did not observe significant correlation between linear CCDC7 expression and circCCDC7(15, 16, 17, 18, 19) (FIG. 2C). Experimentally, we collected 23 pairs of clinical PCa samples. RT-qPCR demonstrated that circCCDC7(15, 16, 17, 18, 19) was indeed expressed at lower levels in tumor samples than in the matched normal margins (FIGS. 2D and 11F). In contrast, linear CCDC7 has no such a trend (FIG. 2E). Unsurprisingly, linear CCDC7 expression had no statistically significant correlation with circCCDC7(15, 16, 17, 18, 19) in our clinical samples, either (FIG. 2F). In addition, the expression of circCCDC7(15, 16, 17, 18, 19) was detected at much higher levels in low Gleason samples (Gleason score=6) in CPGEA, compared to those in the high Gleason samples (Gleason score≥7) (FIG. 2G), consistent with our initial RNA-Seq data. Of note, we also found that circCCDC7(15, 16, 17, 18, 19) was expressed at lower levels in metastasis samples than localized samples (FIG. 11G) in another dataset (GSE99857). These findings support that PCa is associated with reduced expression of circCCDC7(15, 16, 17, 18, 19), and this effect is further increased in accordance with malignancy.

We then divided the clinical PCa cases into two groups according to the junction read counts and found that higher circCCDC7(15, 16, 17, 18, 19) expression predicted a better outcome (FIG. 2h), yet the linear CCDC7 had no correlation with PCa prognosis (FIG. 2i). We also queried the TCGA prostate cancer study to replicate our findings. We could not detect the circular RNA with AGREP in TCGA prostate cancer RNA-Seq, which we presume is due to the polyA-sequencing protocol with TCGA. Additionally, we divided the expression of linear CCDC7 into two groups according to the FPKM value in TCGA, and found that linear CCDC7 had no statistically significant association with prostate cancer patient survival (FIG. 11h). Univariable analysis and multivariable analysis by Cox proportional hazards modeling also demonstrated a similar prognostic value of circCCDC7(15, 16, 17, 18, 19) (Table 6). Consistently, the AUC value of the ROC for tumor or normal was better in circCCDC7(15, 16, 17, 18, 19) (0.703) than that in linear CCDC7(0.534) (FIGS. 11I-11J). Collectively, these results suggest that circCCDC7(15, 16, 17, 18, 19) is regulated differently from linear CCDC7, and may serve as a potential biomarker and/or prognostic marker for PCa patients.

### CircCCDC7(15, 16, 17, 18, 19) Inhibits the Viability, Migration, and Invasion of PCa

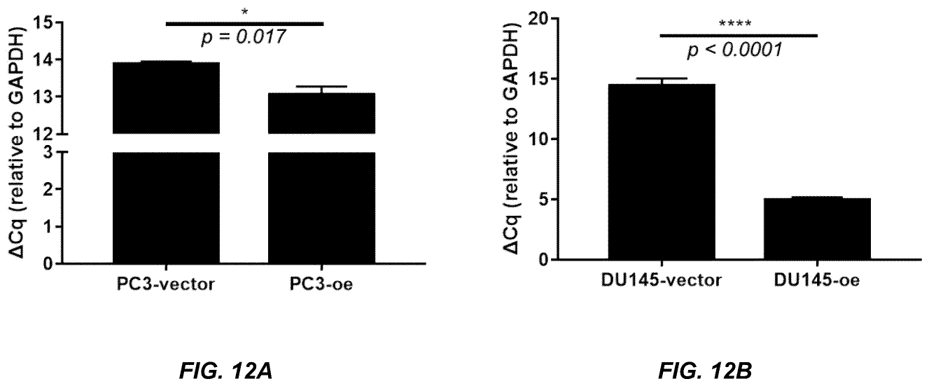

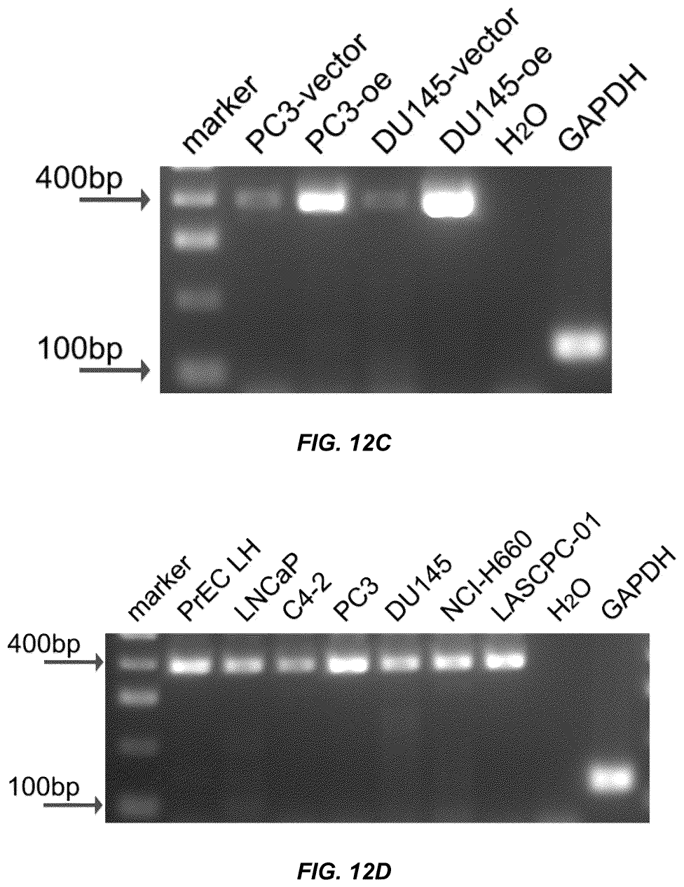

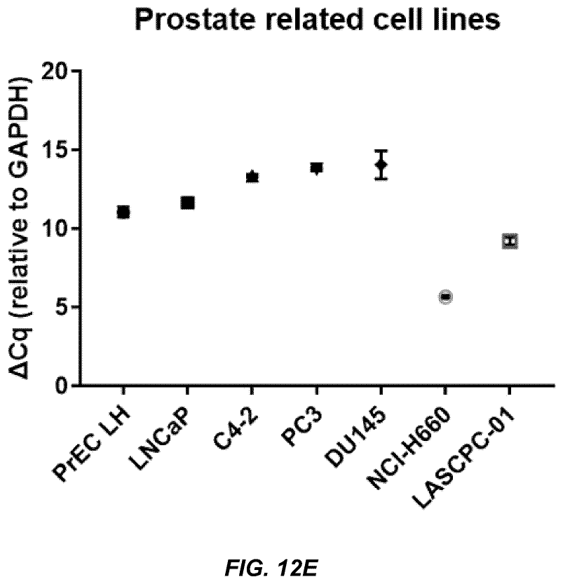

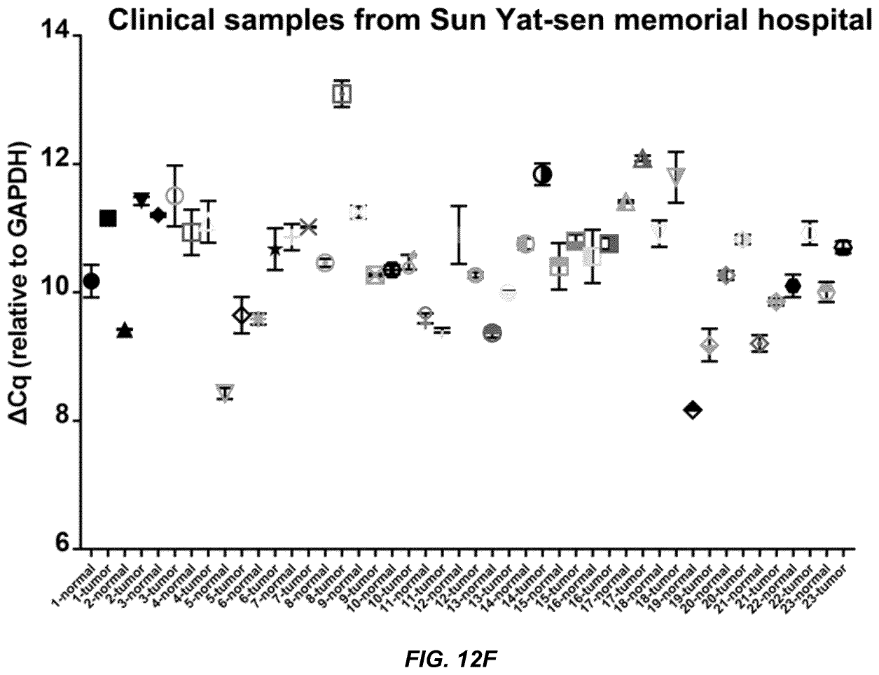

To investigate the effect of circCCDC7(15, 16, 17, 18, 19) on prostate cancer, DU145 and PC3 cell lines were infected with a lentivirus expressing the circular RNA to establish stable circCCDC7(15, 16, 17, 18, 19) overexpressing cell lines. We first confirmed the overexpression system through RT-qPCR and agarose electrophoresis (FIGS. 12A-12C). The ΔCq in PC3 and DU145 overexpression system were about 13 and 5 respectively. To test if the overexpressed circCCDC7(15, 16, 17, 18, 19) levels were still in the range of natural samples, we subsequently performed RT-qPCR on prostate cell lines including PrEC LH, LNCaP, C4-2, PC3, DU145, NCI-H660 and LASCPC-01. All the cells above had a certain degree of circCCDC7(15, 16, 17, 18, 19) expression (FIG. 12D), and the range of ΔCq were from 5 to 15 (FIG. 12E). In addition, the ΔCq in the 23 pairs clinical samples ranged from 8 to 13 (FIG. 12F). The results above suggested the overexpressed circCCDC7(15, 16, 17, 18, 19) levels were still in the range of natural samples.

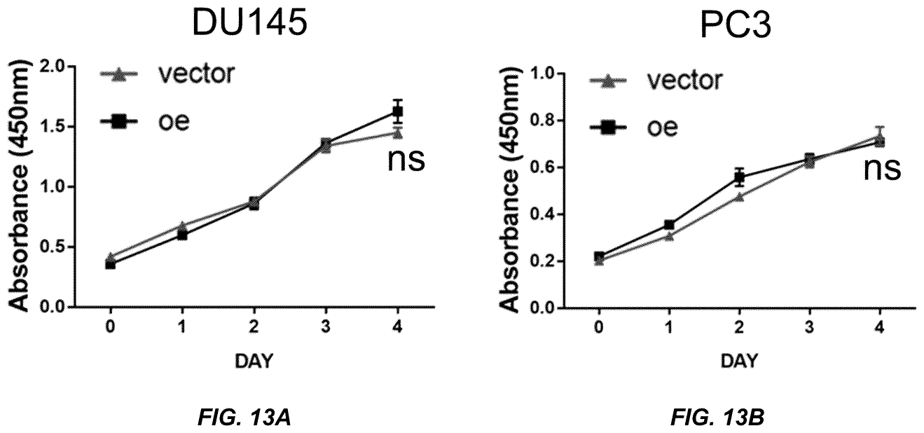

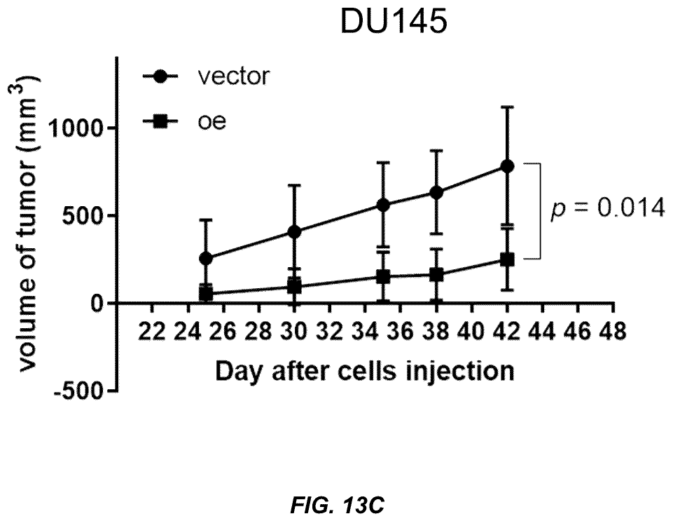

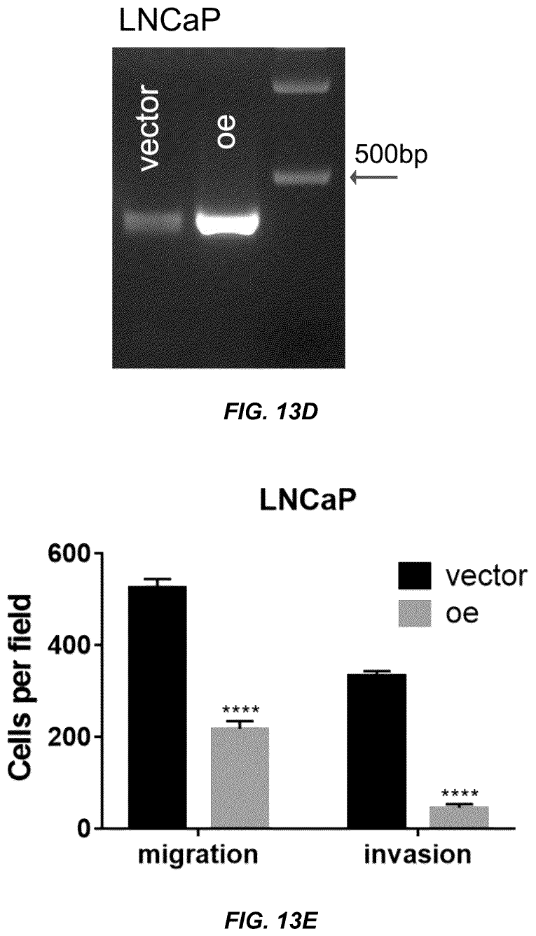

Despite that CCK8 assays did not detect the effect of circCCDC7(15, 16, 17, 18, 19) on cell proliferation of PC3 and DU145 (FIG. 13A-13B), colony formation assays demonstrated its inhibitory effect on cell viability (FIG. 3A). To confirm whether circCCDC7(15, 16, 17, 18, 19) plays an important role in tumorigenesis in vivo, we tested the effect of overexpressed circCCDC7(15, 16, 17, 18, 19) in a dorsal subcutaneous xenograft model. After six weeks, all animals in the control group had reached significant tumor volumes. In contrast, the size and weight of tumors derived from the circCCDC7(15, 16, 17, 18, 19) overexpression group were significantly reduced (FIGS. 3B-3D and 13C). Furthermore, transwell assays showed that circCCDC7(15, 16, 17, 18, 19) could suppress the migration and invasion of both DU145 and PC3 cells (FIGS. 3E-3F). Consistently, the Epithelial-mesenchymal transition (EMT) metastasis promoting marker Slug was down-regulated, EMT suppressive genes E-cad and Claudin were up-regulated with circCCDC7(15, 16, 17, 18, 19) overexpression (FIG. 3G). Additionally, the androgen-dependent LNCaP cell line was also transfected with overexpression plasmid, and transwell assays showed that circCCDC7(15, 16, 17, 18, 19) could suppress the migration and invasion of LNCaP, suggesting that the mechanism of inhibiting PCa is not dependent on AR status (FIG. 13D-13E). Taken together, circCCDC7(15, 16, 17, 18, 19) inhibits the viability, migration, and invasion of PCa in vitro and in vivo.

### CircCCDC7(15, 16, 17, 18, 19) Encodes a Secreted Protein

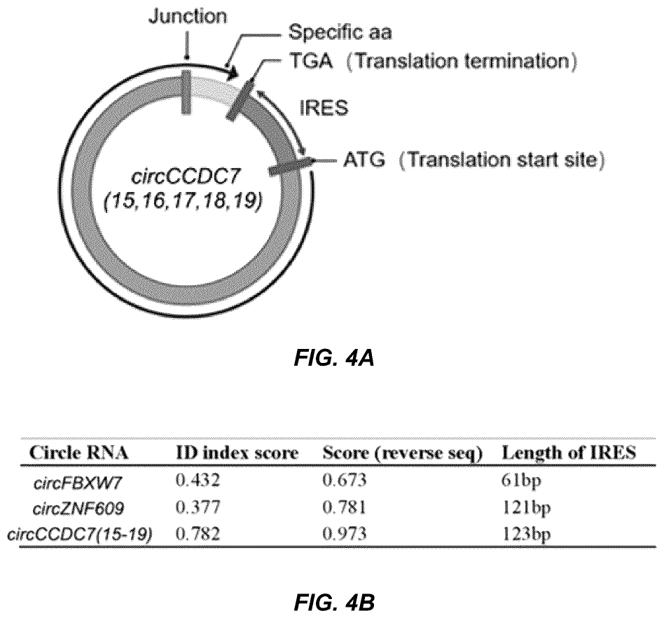

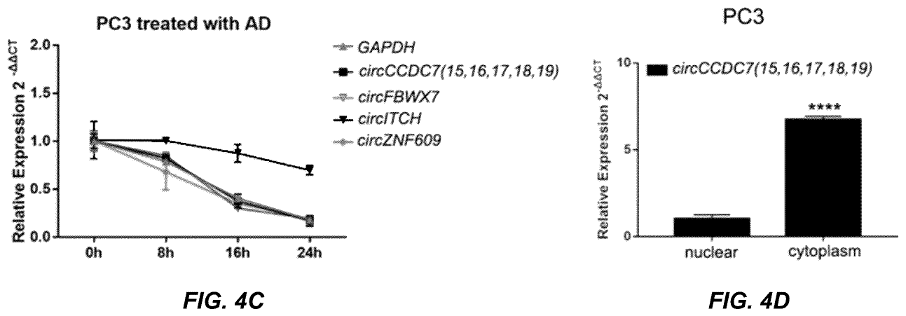

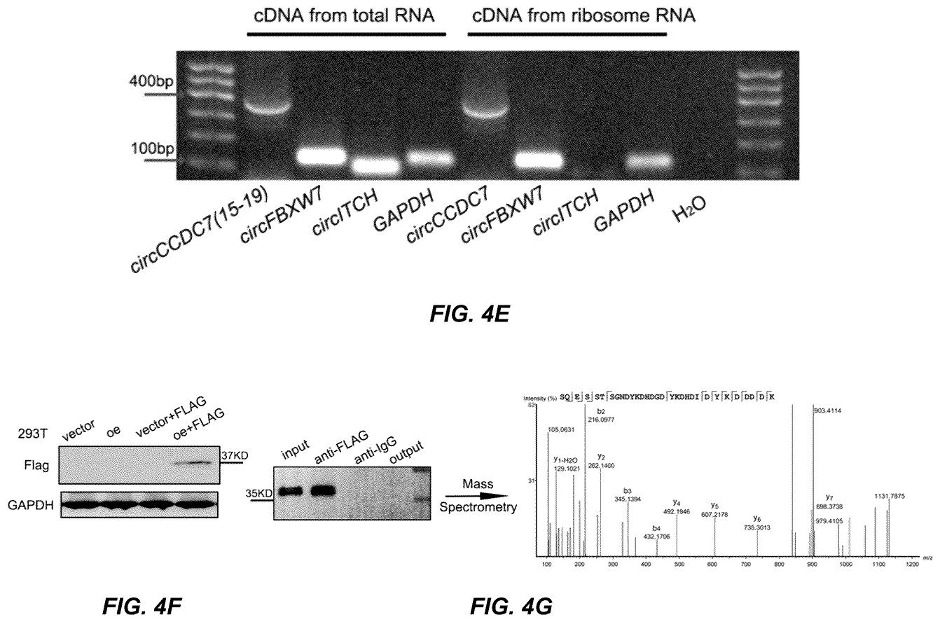

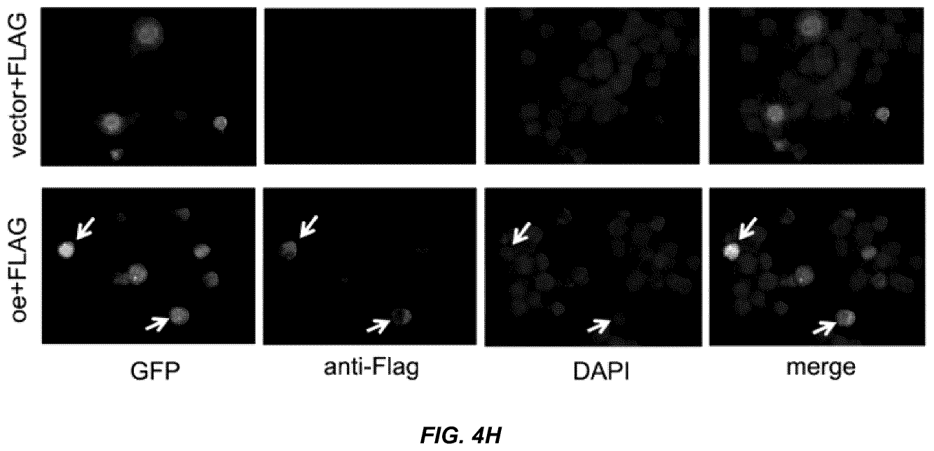

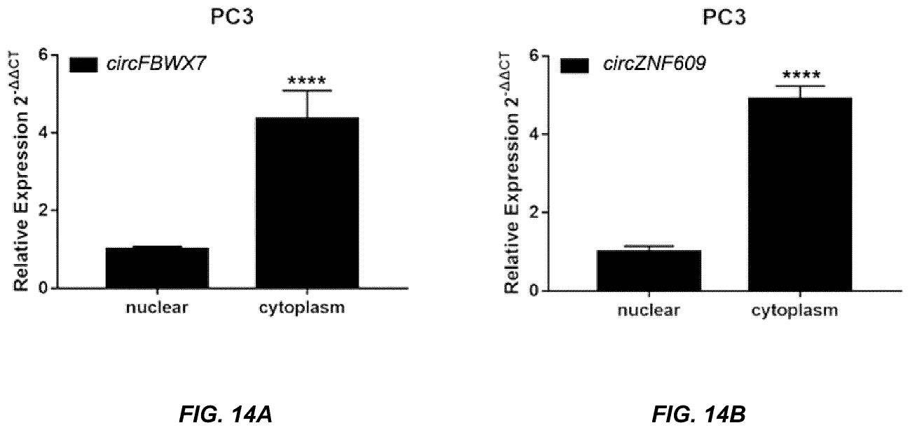

Current knowledge is that circular RNAs work mainly through the following three mechanisms: miRNA sponging, protein binding, and cap-independent translation (Tucker D, et al. World J Clin Oncol. 2020 11(8): 563-572). Protein binding and miRNA sponging related circular RNAs mainly function as ncRNA (non-coding RNA). In contrast, cap-independent translation related circular RNAs mainly rely on the polypeptides/proteins they encode. We here found circCCDC7(15, 16, 17, 18, 19) shared many common features with protein coding circular RNAs. Firstly, after putative open reading frame (ORF) analysis, we found circCCDC7(15, 16, 17, 18, 19) has the basic structure for protein coding, including an internal stop codon (TGA) after junction site, an internal start codon (ATG), and the internal ribosome entry site (IRES) between the stop codon and the start codon (FIG. 4A). Secondly, after IRES analysis, we found the ID index score of IRES in circCCDC7(15, 16, 17, 18, 19) was much higher than that in other protein coding circular RNAs such as circFBXW7 or circZNF609 (Yang Y, et al. J Natl Cancer Inst. 2018 110(3): 304-315; Legnini I, et al. Mol Cell. 2017 66(1): 22-37.e9), predicts its higher binding potential to ribosomes (FIG. 4B). Thirdly, circular RNAs tend to be more stable. However, an actinomycin D assay showed that the half-life of the circCCDC7(15, 16, 17, 18, 19) was about 12 h, which is similar to that of protein-coding RNAs: GAPDH, circFBXW7 and circZNF609, while the half-life of a traditional circular RNA circITCH working as the sponge of miRNA (Yuan Y, et al. Technol Cancer Res Treat. 2019 January-December: 18:1533033819886867; Guo X, et al. Cell Cycle. 2022 21(15): 1557-1577) exceeded 24 h (FIG. 4c). Furthermore, many ncRNAs tend to reside more in the nucleus regulating transcription. However, nucleo-cytoplasmic separation showed that circCCDC7(15, 16, 17, 18, 19), circFBXW7 and circZNF609 were all enriched in cytoplasm (FIGS. 4D and 14A-14B), consistent with their protein-coding roles. Lastly, since RNA-ribosome complex is necessary for protein translation, we extracted mixed ribosomes from cultured PCa cells (LNCaP, C4-2, PC3 and DU145) and validated the existence of circCCDC7(15, 16, 17, 18, 19), linear mRNA GAPDH and circFBXW7 in the complex, yet the traditional circITCH was not detected (FIG. 4E). Taken together, these results supported that circCCDC7(15, 16, 17, 18, 19) was a protein-coding circular RNA.

Based on ORF analysis, we predicted a 180aa protein (circCCDC7-180aa) encoded by circCCDC7(15, 16, 17, 18, 19). 173aa out of 180aa are the same to linear CCDC7 protein, while the last seven amino acids are uniquely encoded by exon19 looping back to exon15. We further used the specific junction amino acids (SQESSTSGN) BLASTP against UniProtKB/Swiss-Prot database and found no exact match (FIGS. 14C-14D), suggesting that this peptide cannot be derived from a linear transcript of another gene.

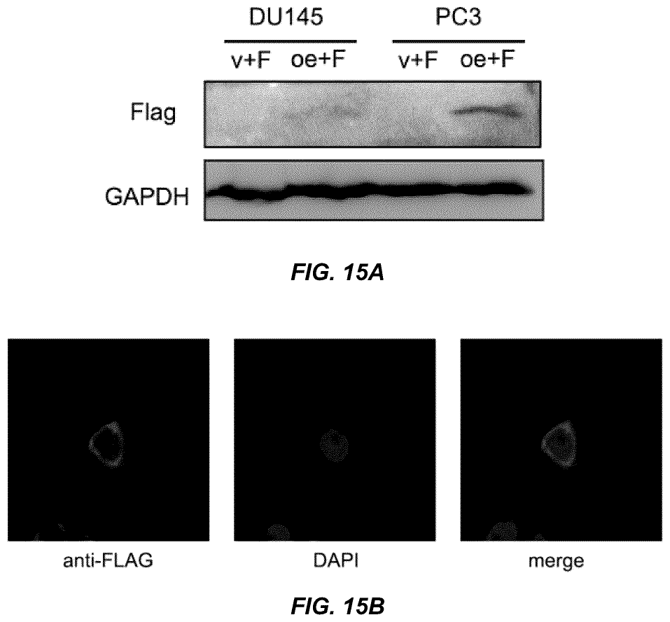

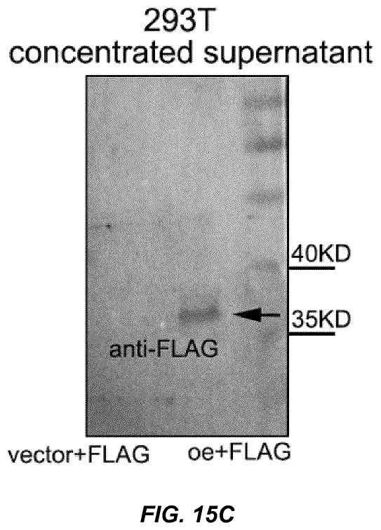

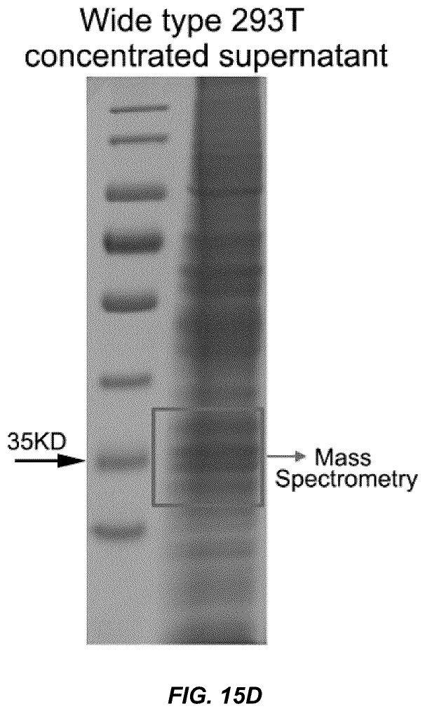

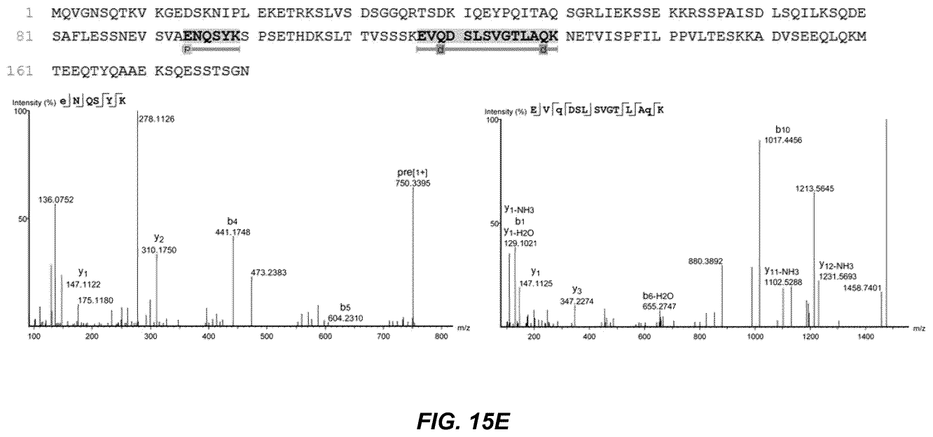

To further confirm the protein-coding potential of circCCDC7(15, 16, 17, 18, 19), we inserted 3×FLAG just before the stop codon and the full-length sequences were cloned to plenti-ciR-GFP-T2A vector plasmid (FIG. 15A). After transient transfection in 293T cells, we detected a clear band around 37kd (FIG. 4F). We could also detect the same bands in PC3 and DU145 despite their relatively lower transfection efficiency (FIG. 15B). Importantly, circCCDC7-180aa was further confirmed by immunoblotting after IP and mass spectrometry, which identified the junction-specific peptides (SQESSTSGN, SEQ ID NO:19) (FIG. 4G). Immunofluorescence was conducted to visualize the expression of the protein. We chose to use HCT116 cells because its higher transfection efficiency and cell shape. The results showed that circCCDC7-180aa was mainly located around nucleus resembling Golgi localization (FIG. 4H). Further immunofluorescence also confirmed that circCCDC7-180aa mainly positioned around the nucleus in the stable overexpression DU145 (FIG. 15C). We thus suspected that circCCDC7-180aa is processed in Golgi and subsequently transferred to cell membrane. To this end, we further isolated membrane from cytosolic fraction, and used Western blot to demonstrate that circCCDC7-180aa was indeed enriched in membrane fraction (FIG. 4I). Because of the localization to Golgi, we further tested whether the protein is also secreted. We first performed an ELISA assay on cell culture supernatant, and the results verified our conjecture (FIG. 4J). Secondly, we harvested the supernatant, and after lyophilization, we detected the correct sized band on a Western blot (FIG. 15D). The following mass spectrometry also identified the peptides of circCCDC7-180aa (FIG. 15E-15F). In summary, we found evidence that circCCDC7(15, 16, 17, 18, 19) encodes a protein with partial novel sequence, which is readily found in cell culture supernatant, suggesting that it is secreted.

### CircCCDC7(15, 16, 17, 18, 19) Suppresses the Progression of PCa by its Encoded Protein

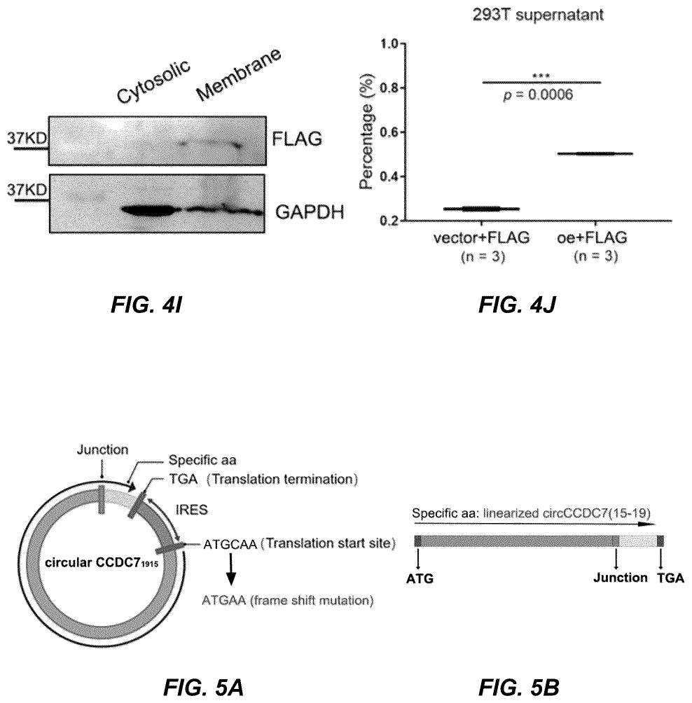

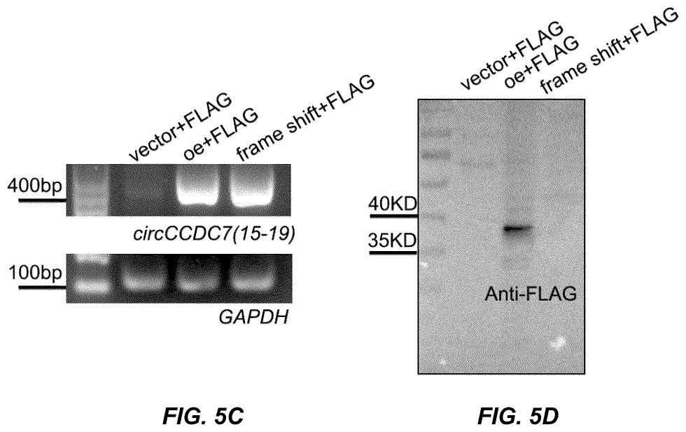

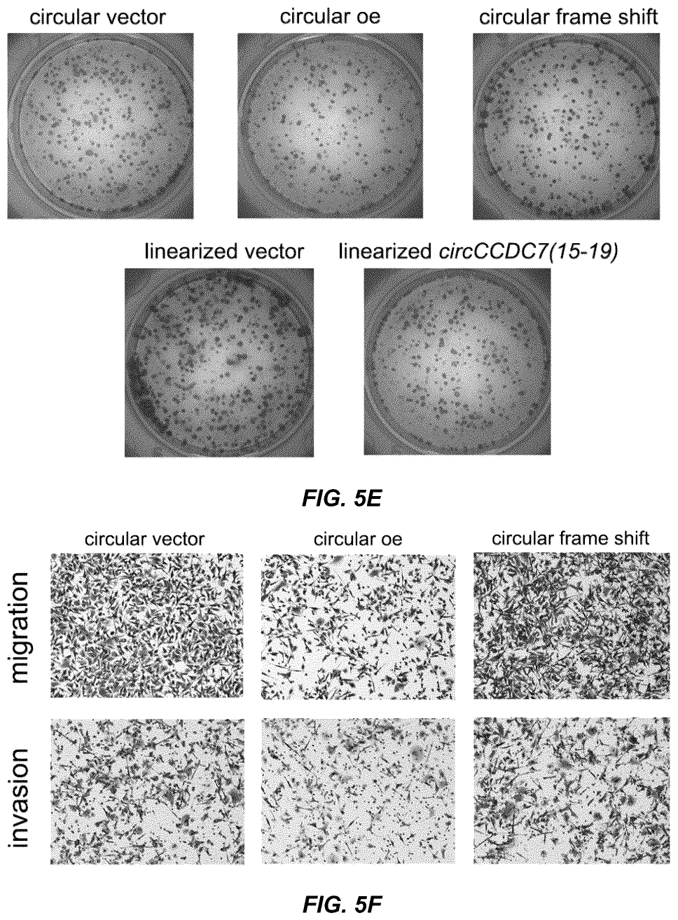

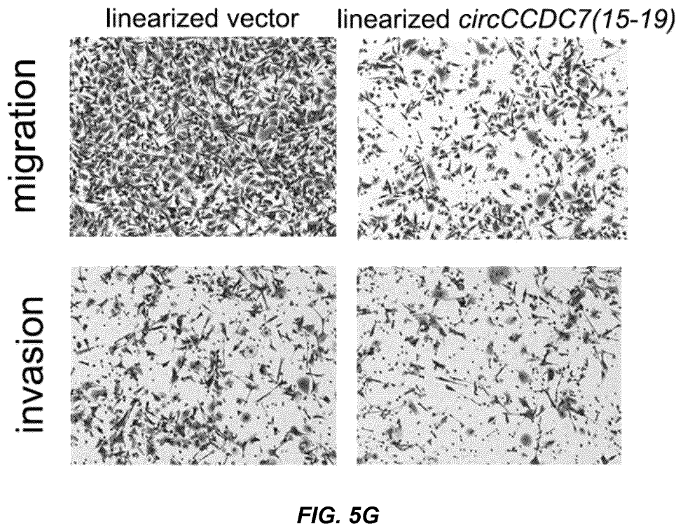

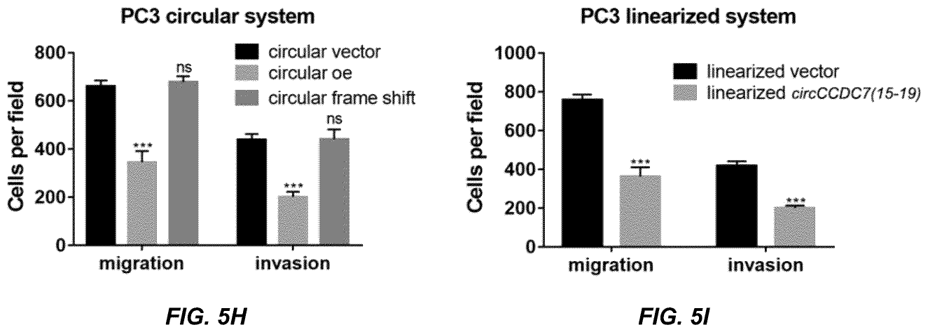

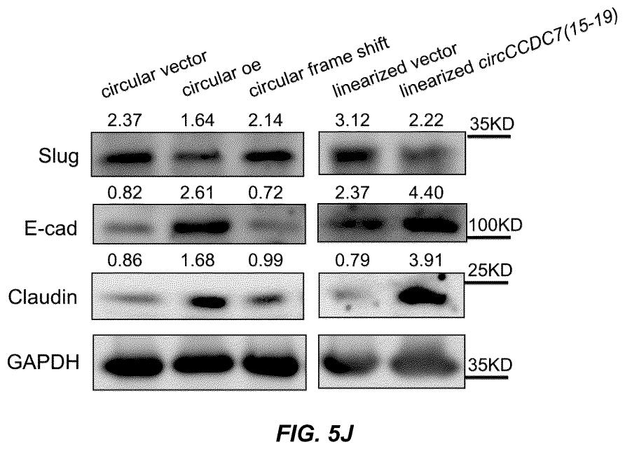

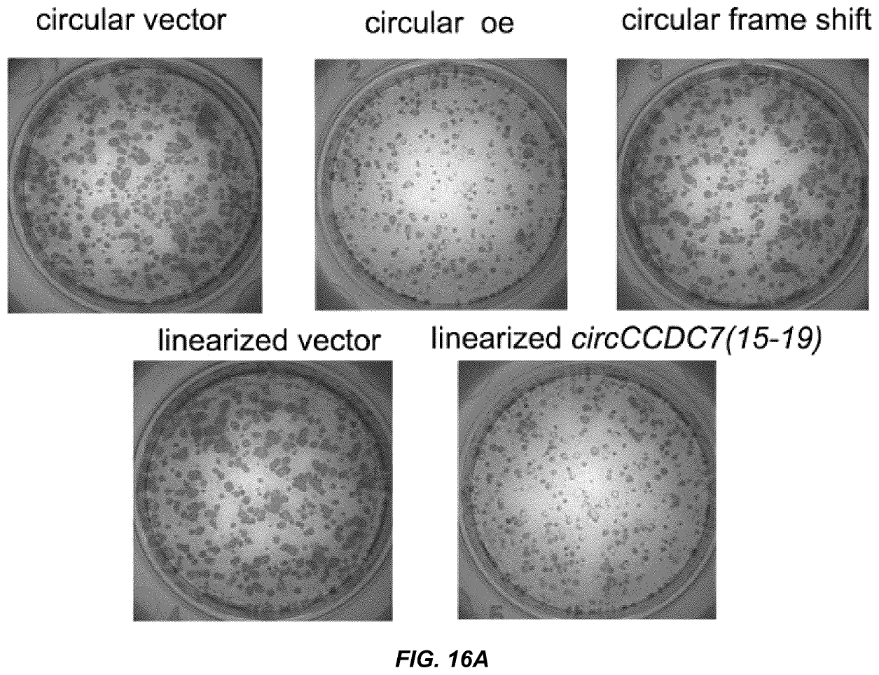

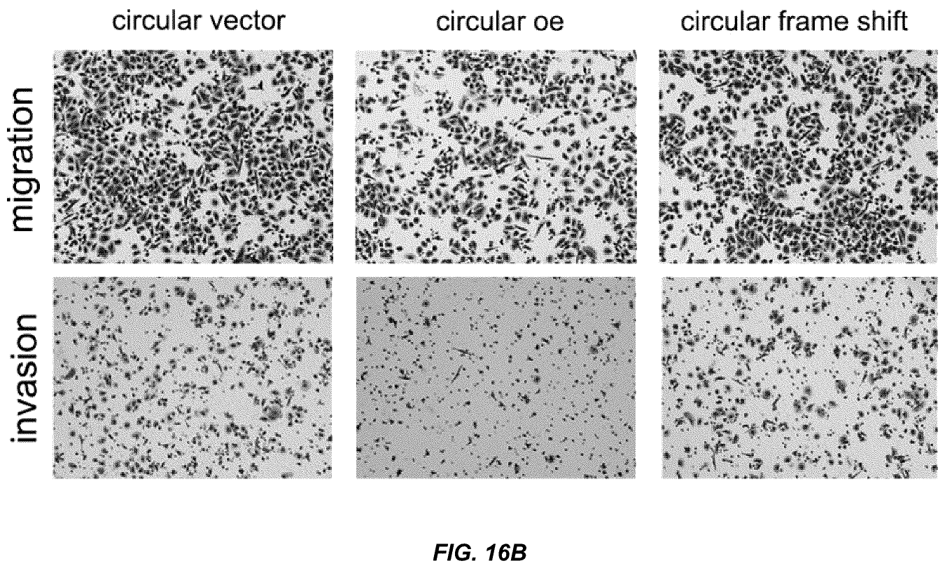

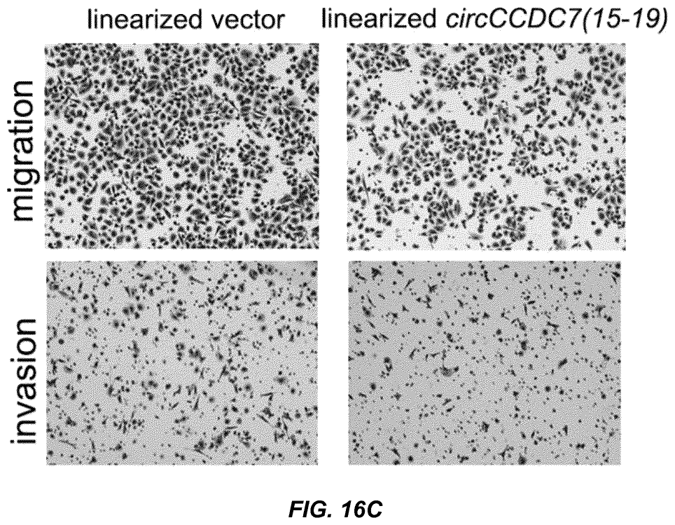

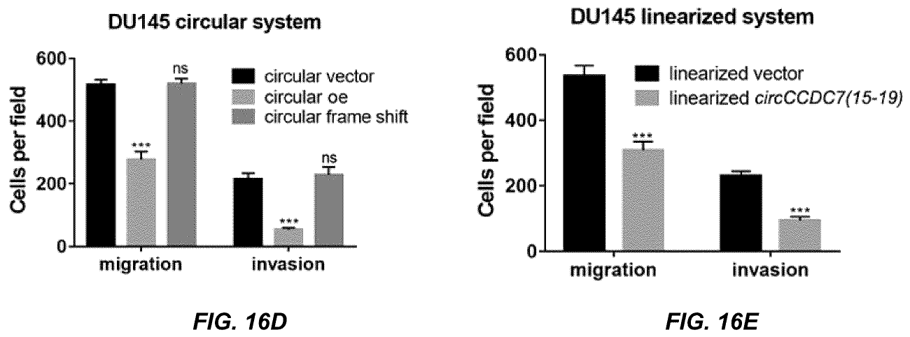

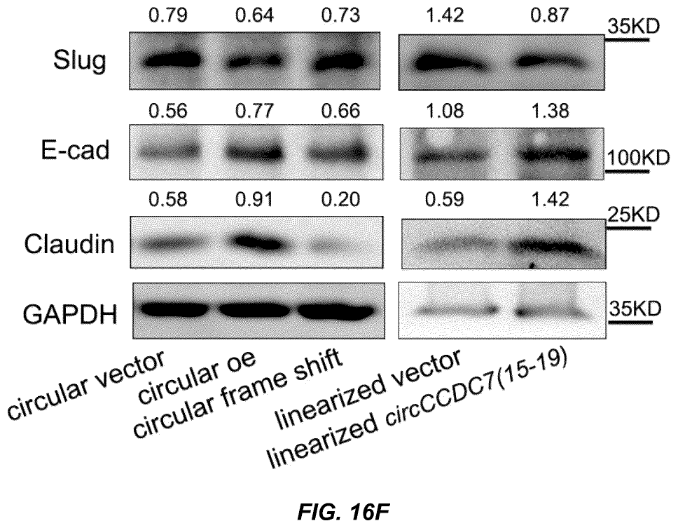

Although some protein-coding circular RNAs have been discovered in recent years, such as circFBXW7 (Yang Y, et al. J Natl Cancer Inst. 2018 110(3): 304-315) and circZNF609 (Legnini I, et al. Mol Cell. 2017 66(1): 22-37.e9), some other studies have demonstrated that they can also exert their functions by acting as miRNA sponges (Du S, et al. Cancer Manag Res. 2020 12:7487-7499; Xu Y, et al. Neoplasma. 2021 68(1): 108-118). Therefore, we felt the need to determine the specific mechanism by which circCCDC7(15, 16, 17, 18, 19) affects the progression of PCa. To distinguish the potential role of as a ncRNA vs. protein coding, we mutated the plasmid of circCCDC7(15, 16, 17, 18, 19) by deleting one base after the start codon (frame shift mutation) (FIGS. 5A and 15G), which presumably will not affect the putative ncRNA activity. As a control, we cloned the same ORF plus 3×FLAG tag into a linear vector (FIGS. 5B and 15H). Further qPCR and agarose electrophoresis validated that circCCDC7(15, 16, 17, 18, 19) and mutated circCCDC7(15, 16, 17, 18, 19) was significantly overexpressed in both wild-type and frame shift mutant groups (FIG. 5C), however, we could not detect the signal of FLAG in frame-shifted circCCDC7(15, 16, 17, 18, 19) via Western blot (FIG. 5D). We transfected these plasmids into PC3 and DU145 cells and measured their effect on cell viability, migration, and invasion. The frame shift mutant circCCDC7(15, 16, 17, 18, 19) did not affect the viability of PCa, while both the wild-type circCCDC7(15, 16, 17, 18, 19) and the linear construct decreased the viability significantly (FIGS. 5E and 16A). Similarity, transwell assay demonstrated circCCDC7(15, 16, 17, 18, 19) with frame shift mutation lost its inhibitory function on migration and invasion of PCa (FIGS. 5F, 5H, 16B, 16D). In contrast, both the wild-type circCCDC7(15, 16, 17, 18, 19) and the linear construct decreased the ability of migration, and invasion in PC3 and DU145 (FIGS. 5G, 5I, 16C, 16E). The metastasis related markers also showed a consistent trend (FIGS. 5J and 16F). These results support that the tumor suppressive ability is due to the protein circCCDC7(15, 16, 17, 18, 19) encodes, but not by a ncRNA-mediated mechanism.

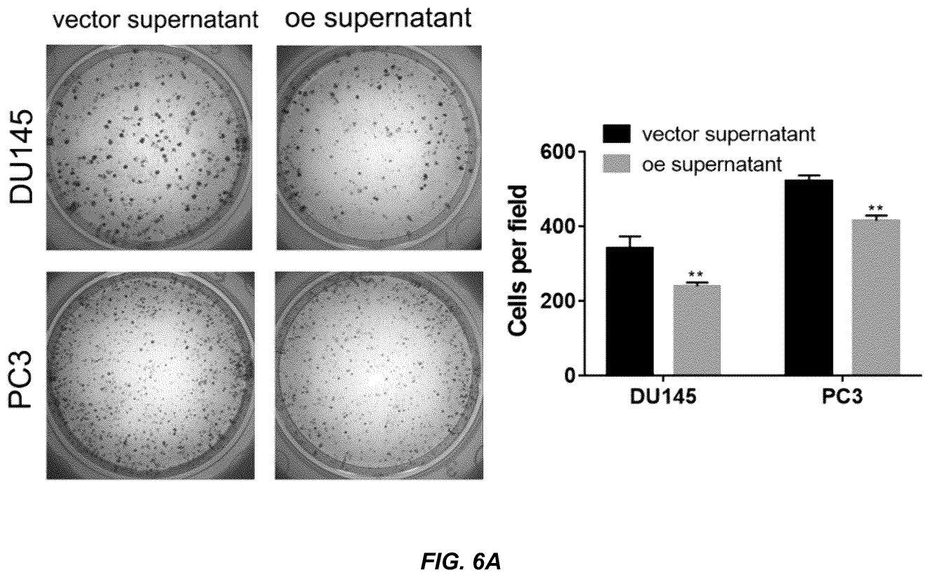

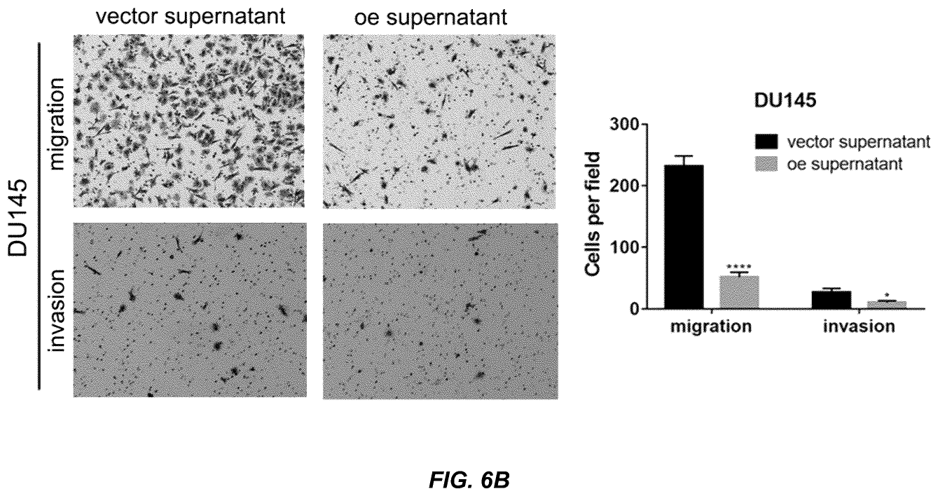

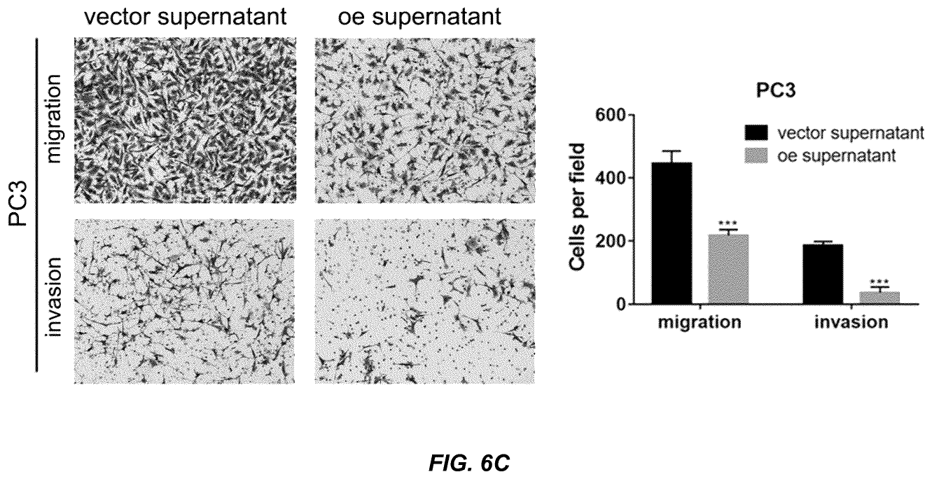

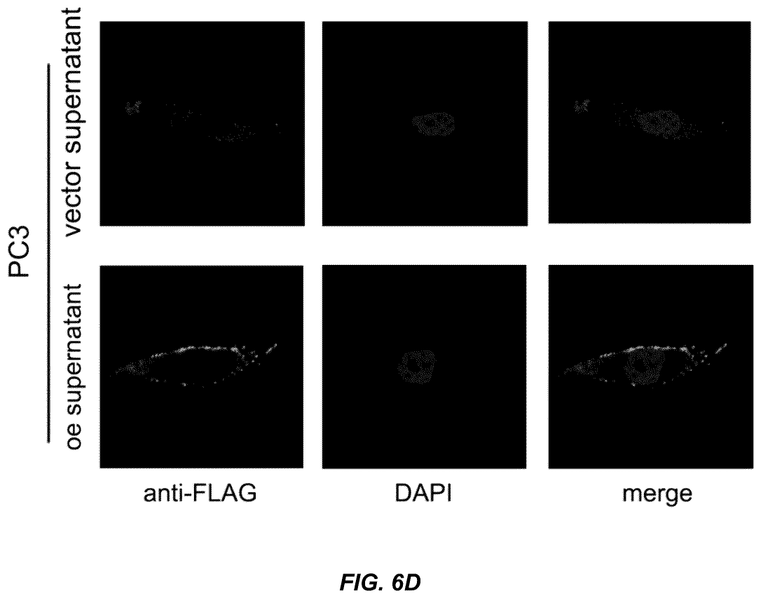

Since we demonstrated that the circCCDC7(15, 16, 17, 18, 19) encoded protein can be secreted outside of the cells, we collected cell media from 293T cells transfected with OE construct of circCCDC7(15, 16, 17, 18, 19). We then added these media to cultured PC3 and DU145. Impressively, a 1:1 ratio of supernatant from circCCDC7(15, 16, 17, 18, 19) OE 293T cells to cell culture media on both PCa cell lines lead to obviously reduced cell viability (FIG. 6A) Similarly, reduced migration and invasion were also observed (FIG. 6B-6C), supporting a potential therapeutic potential for the secreted protein. To visualize the location of the circCCDC7-180aa on recipient cells, the enriched circCCDC7-180aa media from cell media of 293T transfected with FLAG tagged circCCDC7(15, 16, 17, 18, 19) was added to the adherent wide-type PC3 cells. Immunofluorescence confirmed that this protein was mainly enriched on the cell membrane, suggesting that circCCDC7-180aa may regulate the progression of PCa via some receptor-ligand interaction (FIG. 6D).

FLRT3 is a Downstream Target of circCCDC7(15, 16, 17, 18, 19)

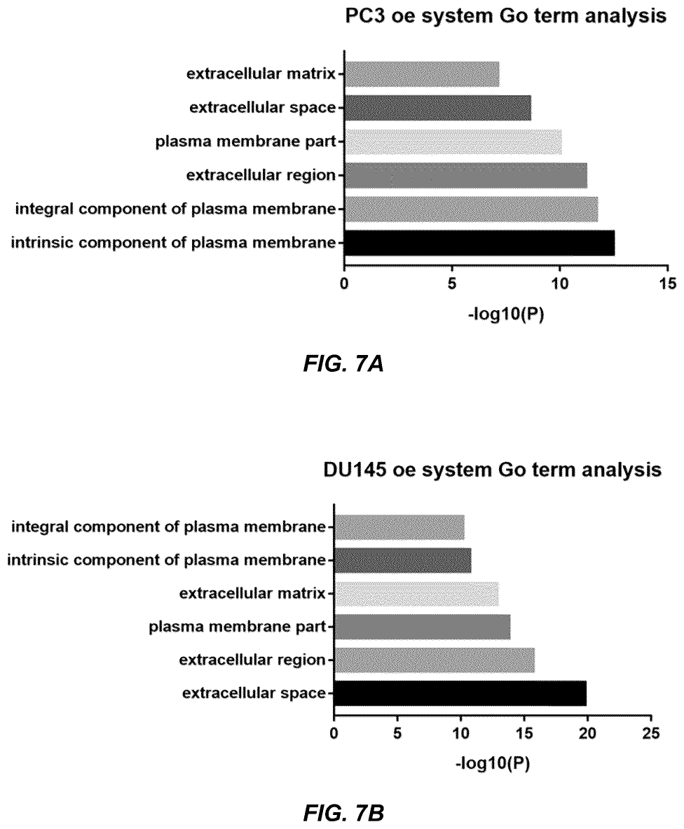

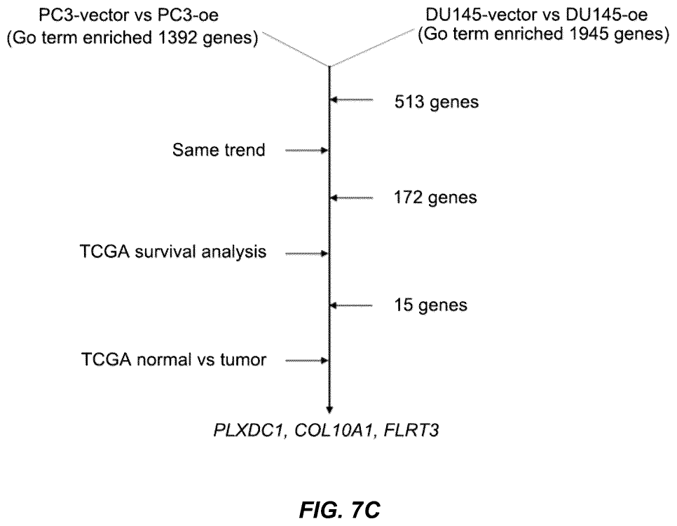

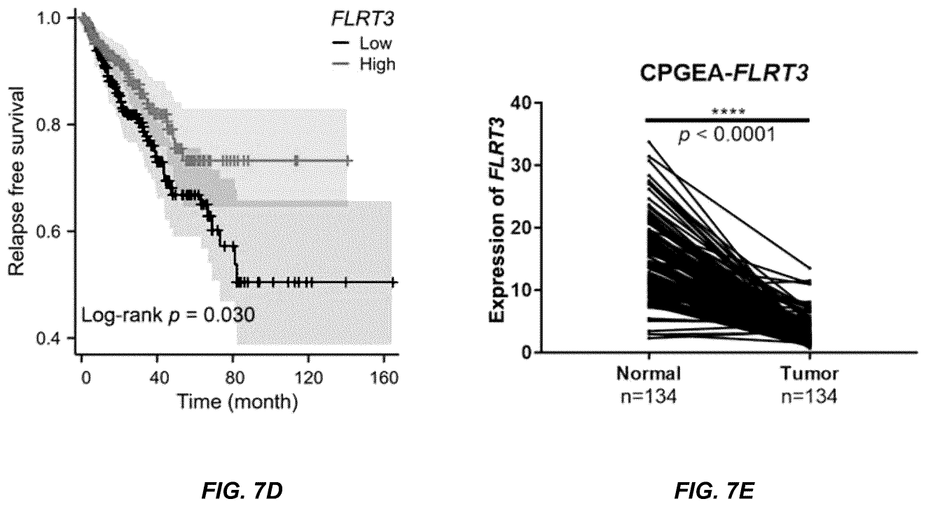

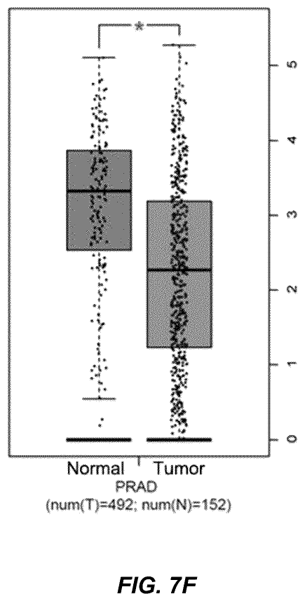

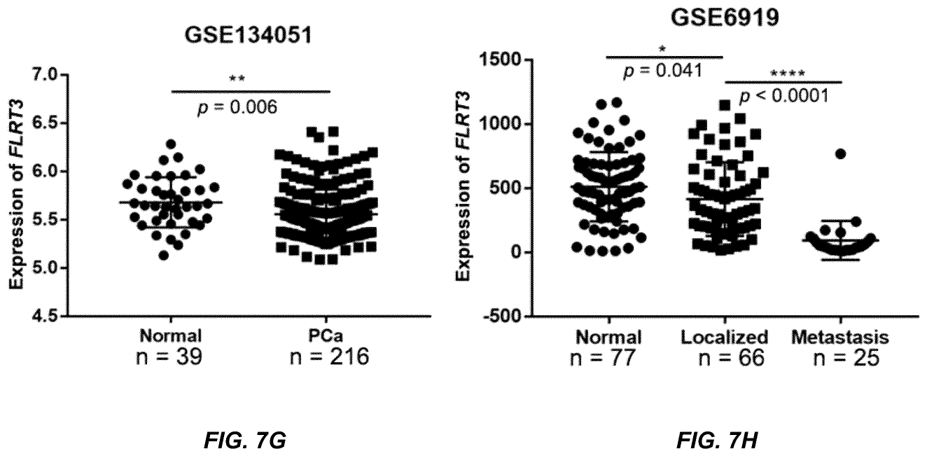

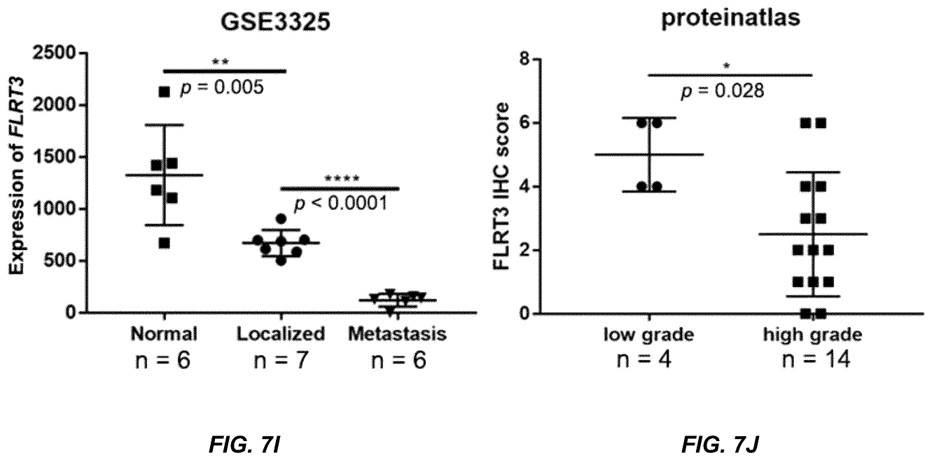

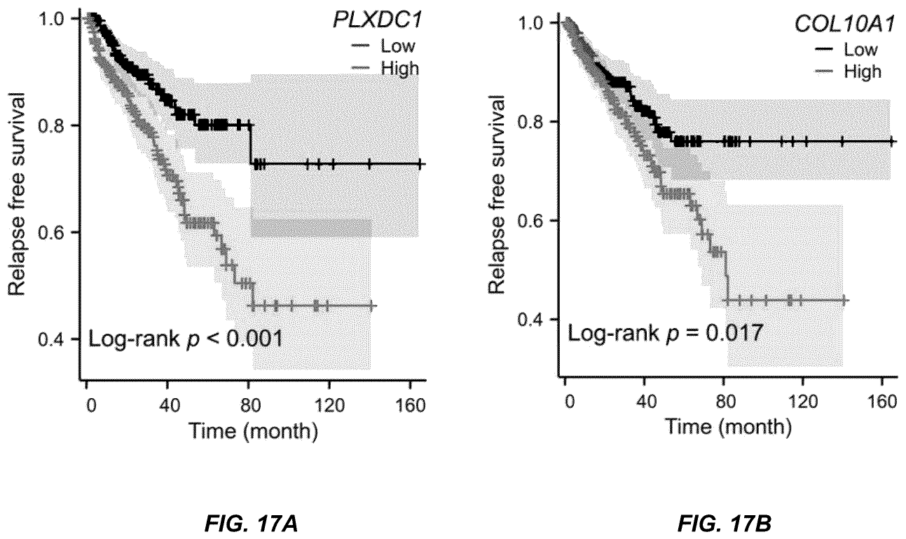

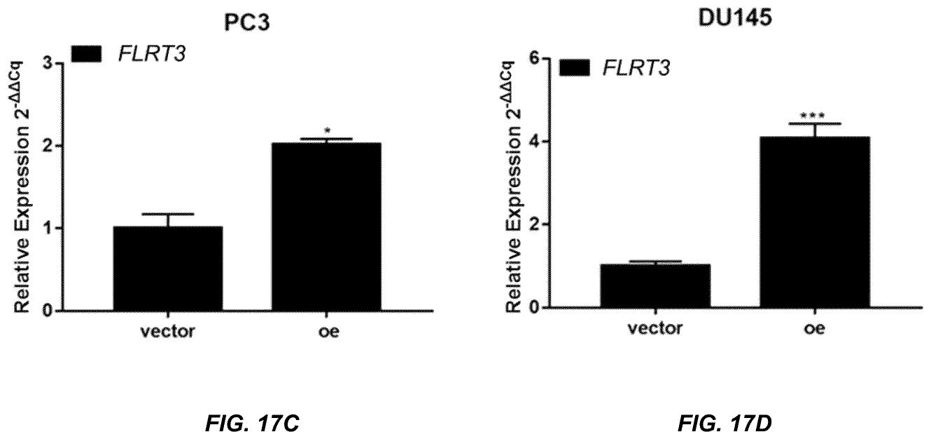

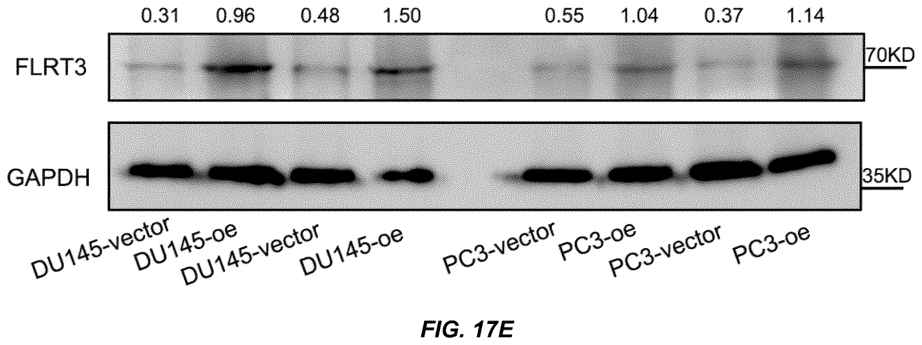

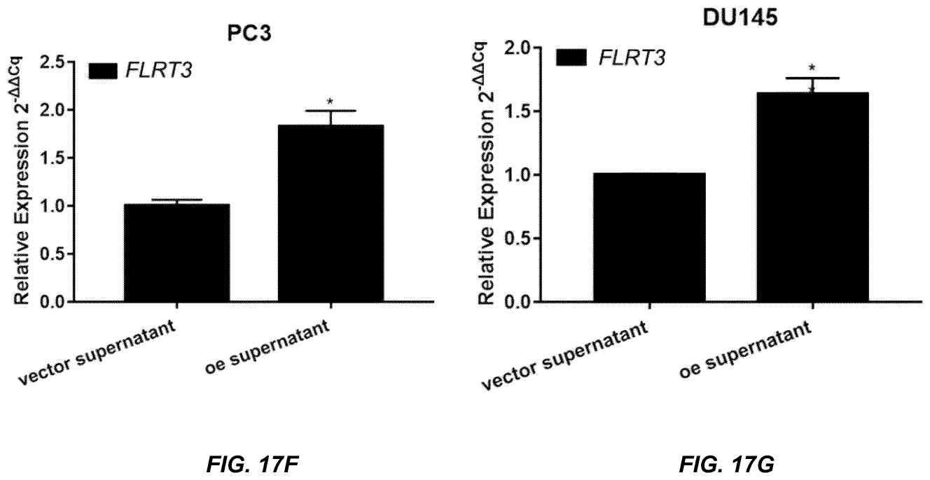

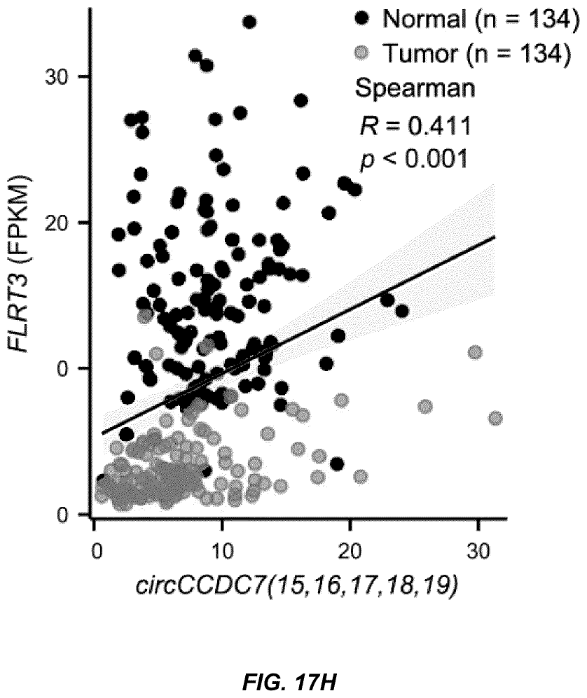

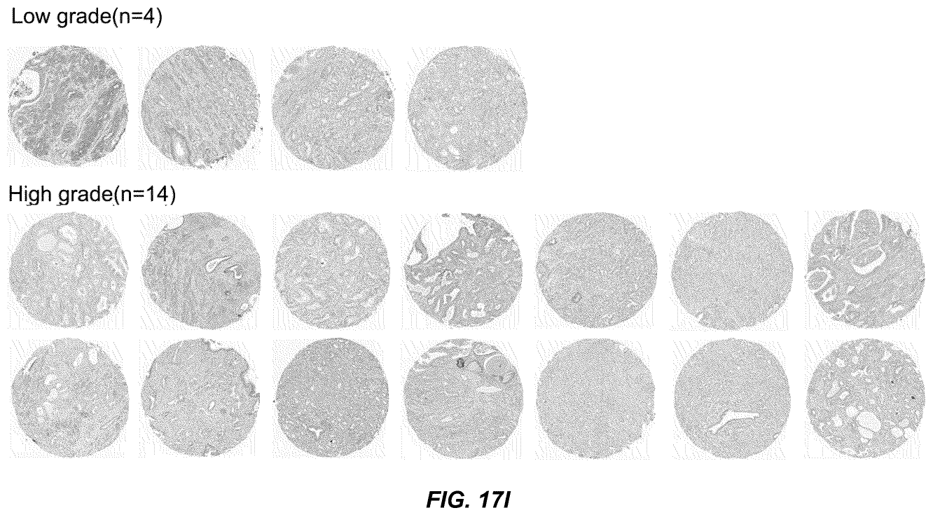

To investigate downstream functional mechanism of circCCDC7(15, 16, 17, 18, 19), next-generation RNA sequencing was performed on PC3 and DU145 cells with circCCDC7(15, 16, 17, 18, 19) overexpression. Genes with log₂|Fold Change|≥1 were considered significantly differentially expressed. Consistent with its role in migration and invasion, we found enriched cellular components important for metastasis, such as extracellular matrix and extracellular region by gene ontology term analysis (FIG. 7A-7B). There are 1,392 GO term enriched genes in PC3 and 1,945 genes in DU145, of which there were 513 overlaps. Among them, 172 genes with the same changing trend in both cell lines were chosen for further analysis. After discarding genes with no association with PCa prognosis, and those with no significant expression difference between tumor and normal tissues in TCGA, three genes remained (FIG. 7C). Among them, PLXDC1 and COL10A1 are both up-regulated after circCCDC7(15, 16, 17, 18, 19) overexpression, but correlated with worse prognosis (FIGS. 17A-17B). In contrast, high expression of fibronectin leucine rich transmembrane protein 3 (FLRT3) (log₂|Fold Change|=1.5 and 3.7, respectively) is correlated with a good prognosis (FIG. 7D). Thus, we decided to focus on FLRT3 as it is most likely to be a downstream mediator of the tumor suppressive activity of circCCDC7(15, 16, 17, 18, 19). Further qPCR and Western blot also validated that circCCDC7(15, 16, 17, 18, 19) indeed upregulates the expression of FLRT3 at RNA and protein level (FIGS. 17C-17E). Consistently, we collected enriched cell media from 293T cells transfected with OE construct of circCCDC7(15, 16, 17, 18, 19). We then added these media to cultured PC3 and DU145 and found up-regulation of FLRT3 (FIGS. 17F-17G). Lastly, we observe positive correlation between FLRT3 expression and circCCDC7(15, 16, 17, 18, 19) in CPGEA dataset (R=0.411, p<0.001) (FIG. 17H).

To explore the role of FLRT3 in PCa, we then compared the RNA level of FLRT3 between normal and tumor samples in CPGEA and TCGA. Similar to circCCDC7(15, 16, 17, 18, 19), FLRT3 was significantly downregulated in tumor samples (FIG. 7E-7F). Analysis with another dataset GSE134051 showed a similar result (FIG. 7G). Importantly, higher mRNA levels of FLRT3 were associated with lower Gleason scores, which is similar to our observation with circCCDC7(15, 16, 17, 18, 19) (Table 7). Since there were few cases of distant metastasis in TCGA and CPGEA, other GEO datasets GSE6919 and GSE3325 were employed to examine the relationship between FLRT3 and tumor metastasis. As expected, the expression of FLRT3 was the lowest in metastases, when compared with localized and normal samples (FIG. 7H-7I). In addition to mRNA level comparisons, we further analyzed FLRT3 protein level in The Human Protein Atlas, which contains the IHC pictures from 18 patients with different grades (FIG. 17I). The immunoreactivity score was calculated as the intensity score multiplied by the quantity score (Table 8), and the results showed that at protein level, higher level of FLRT3 was correlated with lower PCa grades (FIG. 7J). Taken together, we hypothesized circCCDC7(15, 16, 17, 18, 19) regulates the progress of PCa through FLRT3.

Knocking Down FLRT3 Rescues the Effect of circCCDC7(15, 16, 17, 18, 19) on PCa

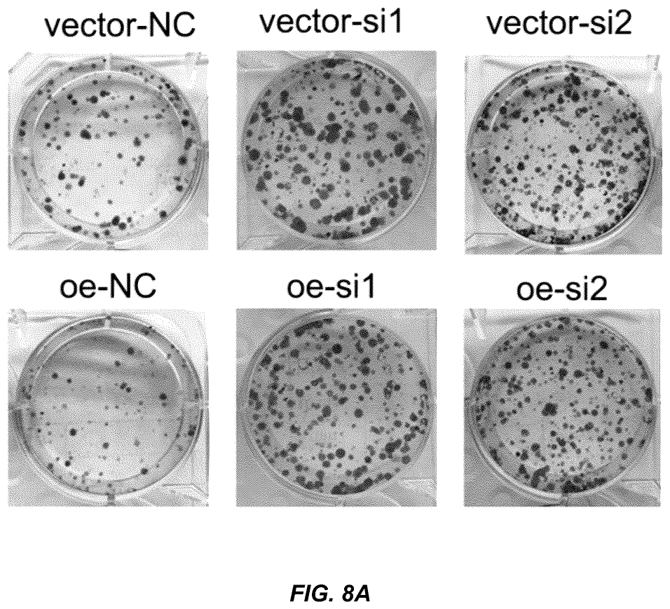

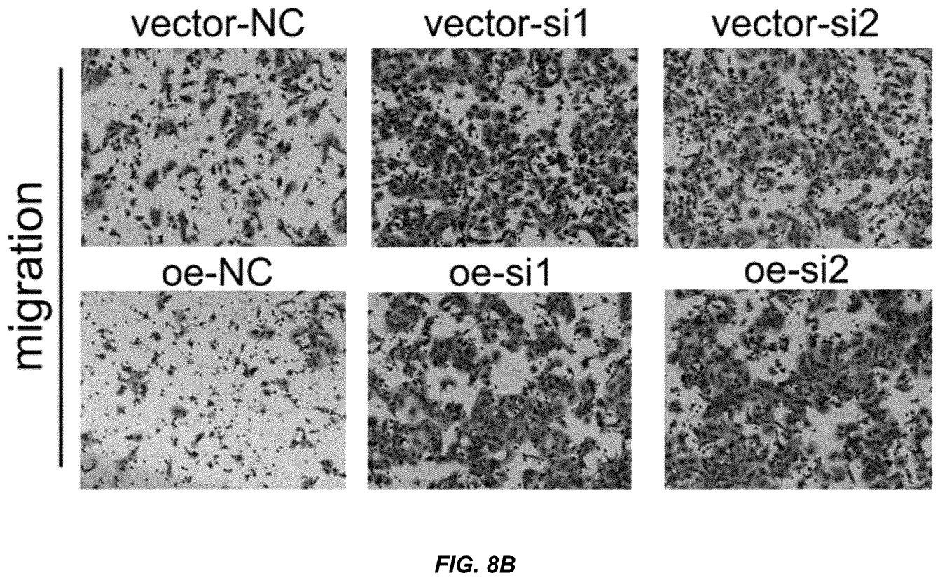

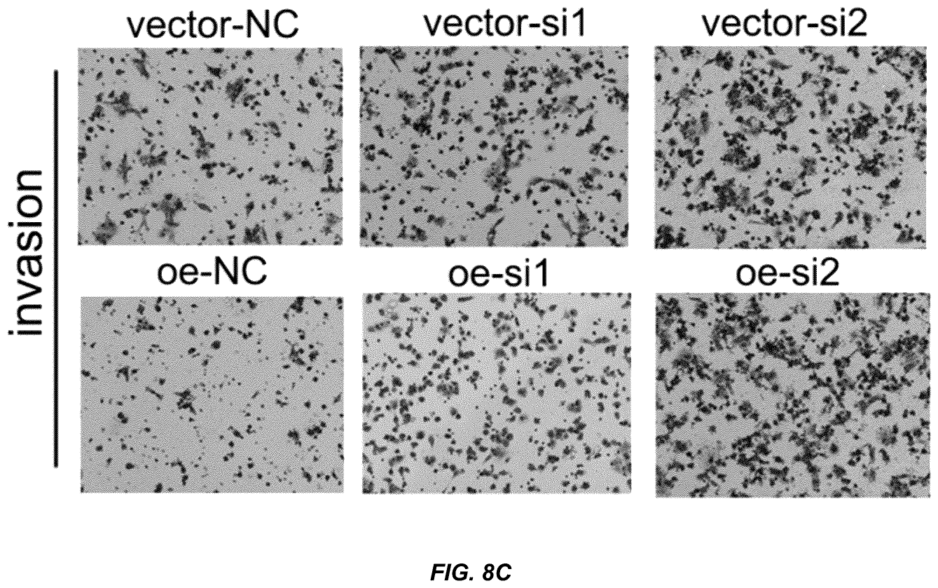

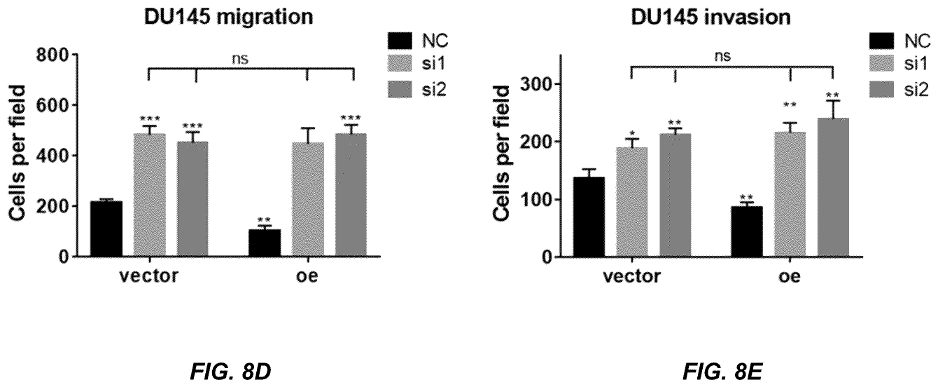

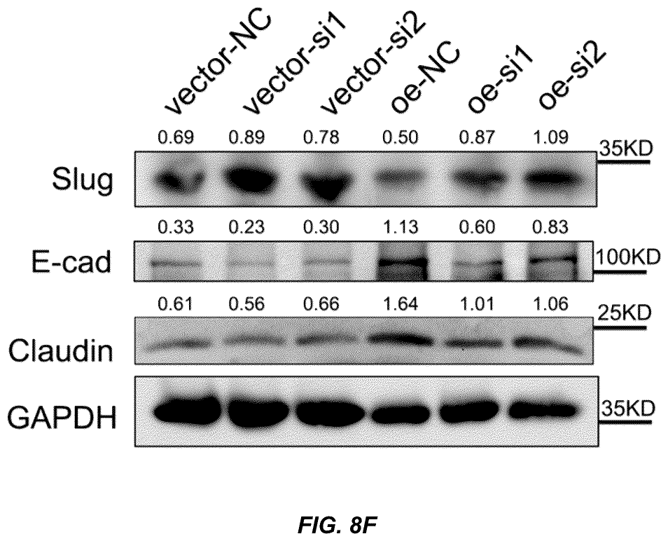

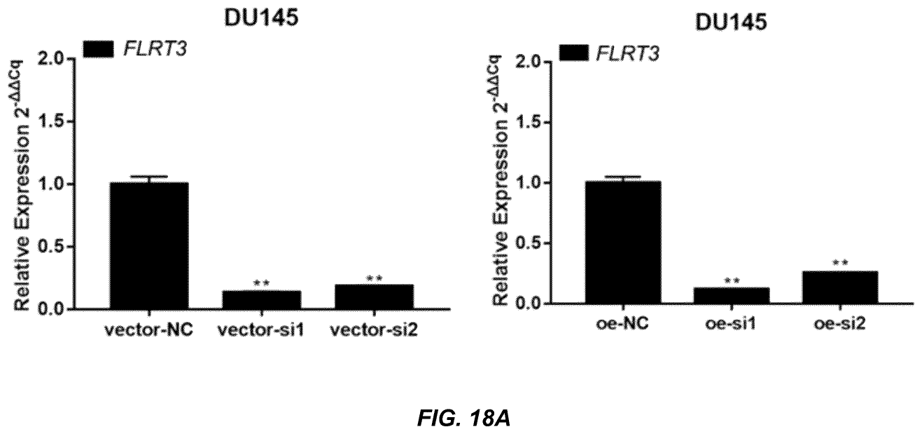

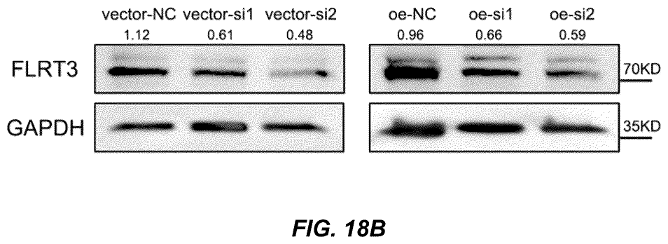

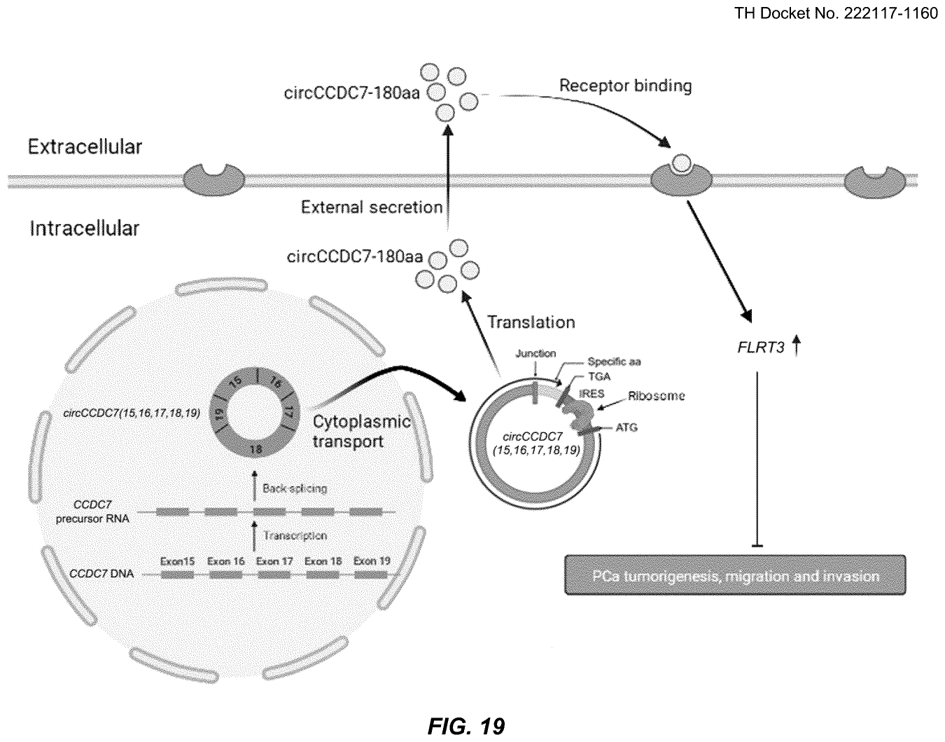

FLRT3 encodes a member of the fibronectin leucine rich transmembrane protein (FLRT) family, which may function in cell adhesion and/or receptor signaling (Chen X, et al. PLOS One. 2009 4(12): e8411). FLRT3 is considered a ligand of neuronal receptor latrophilin 1 (LPHN1) and has been reported to be involved in the immune escape of breast cancer cells of different origins (Yasinska I M, et al. Front Immunol. 2019 10:1594). However, there has been no reports regarding its role in prostate cancer. We overexpressed circCCDC7(15, 16, 17, 18, 19) and then used siRNAs to knockdown the expression of FLRT3 in PCa cells. qPCR and Western blot validated the knocking down efficiency in vector and circCCDC7(15, 16, 17, 18, 19) overexpression groups (FIG. 18). Interestingly, reducing the level of FLRT3 could cancel out the suppressive effect caused by circCCDC7(15, 16, 17, 18, 19) on the viability of PCa cells (FIG. 8A). Similarity, the reduced cell migration/invasion caused by circCCDC7(15, 16, 17, 18, 19) can be entirely offset by knocking down FLRT3 (FIG. 8B-8E), and a similar change was found in metastasis related markers Slug, E-cad and Claudin (FIG. 8F), suggesting that FLRT3 functions downstream of circCCDC7(15, 16, 17, 18, 19). All these results indicate that circCCDC7(15, 16, 17, 18, 19) inhibits metastasis and viability of PCa cell at least partially in a FLRT3-dependent manner (FIG. 19).

### Discussion

Compared with other tumors, prostate cancer has high heterogeneity, which poses obstacles for its diagnosis and treatment. This high intratumoral heterogeneity of PCa may manifest in many aspects, including genomics, epigenetics, and phenotype (Wu B, et al. Int J Cancer. 2020 146(12): 3369-3378). Gleason score has long been regarded as the most important evaluation of PCa and is significantly correlated with the outcomes of patients (Vo J N, et al. Cell. 2019 176(4): 869-881.e13). We here used a series of screening criteria, and finally chose tumor samples from different lobes with different Gleason scores from the same patients. We believe that in depth analysis of these samples will enhance our understanding of the heterogeneity of prostate cancer.

In this study, we found circCCDC7(15, 16, 17, 18, 19) specifically expressed in low Gleason samples. It should be mentioned that using AGREP, we failed to detect circCCDC7(15, 16, 17, 18, 19) in TCGA dataset possibly due to its polyA sequencing platform. However, we successfully found reads supporting its existence in other datasets where the total RNA sequencing was performed, suggesting that total RNA sequencing is necessary to discover this circular RNA. Additionally, the circRNA sequences in public database were often only predicted and sometimes contain introns. Thus, it is possible that circCCDC7(15, 16, 17, 18, 19) we found here is the same circular RNA has been discovered before as hsa_circ_0008679.

Circular RNAs have been promoted as cancer diagnostic markers and therapeutic targets (Vo J N, et al. Cell. 2019 176(4): 869-881.e13; Wang J, et al. Cancer Biol Med. 2021 18(2): 421-436), and interest in the machinery that drives their genesis and function has intensified over the last few years. Until now, most studies demonstrated that circular RNAs exert their regulatory functions through miRNA sponging or protein binding (Taheri M, et al. Front Oncol. 2021 11:781414). Here we accumulated multiple lines of evidence supporting that circCCDC7(15, 16, 17, 18, 19) encodes a protein, which is the mechanism of action for its tumor suppressive activity: 1) by analyzing its sequence, we found a strong IRES sequence between the stop and start codon on the circle; 2) in contrast to other circular RNAs that are more stable, circCCDC7(15, 16, 17, 18, 19) has a relatively short half-life similar to other protein-coding circRNAs; 3) circCCDC7(15, 16, 17, 18, 19) mostly resides in cytosolic fraction, consistent with its protein coding role, not the usual ncRNA role in regulating transcription in nucleus; 4) circCCDC7(15, 16, 17, 18, 19) is enriched in ribosomal-enriched RNA fractions; 5) circular RNA expression constructs with in frame FLAG resulted in protein expression confirmed by Western blot and mass spectrometry analysis; and 6) conversely, a frame shift mutant with only one base deletion at the start codon failed to function, whereas a linear ORF expression construct behaved the same as circCCDC7(15, 16, 17, 18, 19). Of note, a given circular RNA may work through many different mechanisms. For example, circFBXW7 attenuates malignant progression in lung adenocarcinoma by sponging miR-942-5p, as well as encoding FBXW7-185aa to repress glioma tumorigenesis (Yang Y, et al. J Natl Cancer Inst. 2018 110(3): 304-315; Dong Y, et al. Transl Lung Cancer Res. 2021 10(3): 1457-1473). Therefore, it will not be surprising if circCCDC7(15, 16, 17, 18, 19) exerts its function through different mechanisms in different situations.

It is also interesting to notice that among the few examples of circRNAs which encode proteins, most have anti-tumor effects: circLINC-PINT has been reported to produce PINT87aa that suppresses glioblastoma (Zhang M, et al. Nat Commun. 2018 9(1): 4475); FBXW7-185aa produced from circFBXW7 mentioned above is known to inhibit cancer proliferation and migration (Ye F, et al. Mol Ther Nucleic Acids. 2019 18:88-98); circPPP1R₁₂A-73aa from circPPP1R₁₂A was found to display a tumor suppressive role in colon cancer (Zheng, X. et al. Mol Cancer. 2019 18(1): 47). However, most of them cannot be secreted, so the possibility of being a therapeutic agent is greatly reduced. Just like insulin for diabetes, circCCDC7-180aa encoded by circCCDC7(15, 16, 17, 18, 19) supports a new direction for PCa intervention.

Although we have confirmed that FLRT3 is a potential downstream mediator, we did not explore the specific mechanism how circCCDC7(15, 16, 17, 18, 19) regulates FLRT3. FLRT3 is a putative type I transmembrane protein containing 10 leucine-rich repeats, a fibronectin type III domain, and an intracellular tail (Tsuji L, et al. Biochem Biophys Res Commun. 2004 313(4): 1086-91), which is supposed to regulate neuronal cell outgrowth and morphogenesis (Robinson M, et al. Mol Cell Neurosci. 2004 27(2): 202-14). Some other studies also found that FLRT3 plays an important role in other biological processes. For example, FLRT3 is reported to be involved in the prevention of anti-tumor immunity via Tim-3-Galectin-9 pathway (Yasinska I M, et al. Front Immunol. 2019 10:1594). Jauhiainen et al., demonstrated FLRT3 has a role in endothelial cells via regulation of VEGF-stimulated EC-survival, migration, and tube formation (Jauhiainen S, et al. Front Physiol. 2019 10:224). Ma et al., showed FLRT3 is a significant and independent prognostic signature for lung squamous cell carcinoma (Ma X, et al. PeerJ. 2020 8: e9086). However, there has been no study related to prostate cancer. We did not detect by immunoprecipitation its interaction with circCCDC7-180aa and (data not shown), suggesting that FLRT3 may be indirectly regulated by circCCDC7(15, 16, 17, 18, 19). It is possible that circCCDC7-180aa acts as a ligand after its extracellular secretion, and then receptor-ligand complexes formed to regulate the expression of FLRT3. Additionally, we believe that the downstream targets of circCCDC7(15, 16, 17, 18, 19) are not just FLRT3. More studies in PCa have revealed the critical role of the tumor microenvironment in the initiation and progression to advanced disease (Shiao S L, et al. Cancer Lett. 2016 380(1): 340-8), in which secreted protein is one of the important tools for communicating between tumor and its microenvironment (Zhang X. Cancer Commun (Lond). 2019 39(1): 76). Therefore, our future research will be committed to the influence of circCCDC7-180aa on other cells in tumor microenvironment, such as tumor-related macrophages (TAMs), cancer-associated fibroblasts (CAFs), and even T cells.

### Methods

### Clinical Samples

Clinical samples were selected and processed as follows: Patients with more than two tumor sites which distributed in at least two separate lobes were selected based on MR reports, and target tumors were dissected according to MR images. Half of each tumor was submitted to the Pathology department to receive a Gleason score evaluation. Three patients with variable Gleason scores, defined as one lobe's Gleason score higher than 4+3, and another lower than 3+4, were selected for further study. We chose a Gleason score of 7 as the cut-off point because the patients with GS<7 (ISUP grade 1) were considered to be low-risk, GS=7 (ISUP grade 2/3) were considered as intermediate-risk, and GS>7 (ISUP grade 4/5) were considered to be high-risk according to internationally recognized EAU guidelines for prostate cancer. A total of six tumor tissues from three patients were obtained, and submitted for RNA-Sequencing (FIG. 9).

Another set of fresh tumor and adjacent normal tissues of 23 PCa patients from Sun Yat-sen Memorial Hospital were obtained for RT-qPCR validation. All fresh samples were immediately snap-frozen in liquid nitrogen and stored at −80° C. until required. The use of tissues and clinical information in this study was approved by the Sun Yat-sen University's Committees for Ethical Review of Research Involving Human Subjects (approval no. SYSEC-KY-KS-2020-201). All patients submitted their written informed consents. We state that we have complied with all relevant ethical regulations including the Declaration of Helsinki.

### Bioinformatics

Three pairs of different Gleason scored PCa tissues were sent for total RNA sequencing (RiboBio Co., Ltd. Guangzhou, China). In detail, total RNA was isolated from tissues using the Magzol Reagent (Magen, China) according to the manufacturer's protocol, The quantity and integrity of RNA yield was assessed by using the K5500 (Beijing Kaiao, China) and the Agilent 2200 TapeStation (Agilent Technologies, USA) separately. Briefly, rRNAs were removed from Total RNA using QIAseq FastSelect-rRNA HRM KIT (QIAGEN, Germany) and fragmented to approximately 200 bp. Subsequently, the purified RNA fragments were subjected to first strand and then the second strand cDNA was synthesized using dUTP following by adaptor ligation and enrichment with a low-cycle according to instructions of NEBNext Ultra Directional RNA Library Prep Kit for Illumina (NEB, USA). The purified library products were evaluated using the Agilent 2200 TapeStation and Qubit (Thermo Fisher Scientific, USA). The libraries were sequenced by Illumina (Illumina, USA) with paired-end 150 bp at Ribobio Co. Ltd (Ribobio, China). EricScript software (version 0.5.5b) was applied using the hg38 reference genome and default parameters to analyze the RNA sequencing raw data and predict chimeric RNAs. We discarded chimeric RNAs with EricScore<0.6. Blat filtering was applied to filter out false positive events followed by further filtering out events matching a list of chimeric RNAs from healthy individuals as described in our previous work (Singh S, et al. Nucleic Acids Res. 2020 48(4): 1764-1778). RNA samples of circCCDC7(15, 16, 17, 18, 19) overexpressed PC3 and DU145 were also sent for polyA RNA sequencing to explore the possible downstream targets. GO term analysis (http://cbl-gorilla.cs.technion.ac.il/) was performed for the joint differential genes between PC3 and DU145 with hg38 background. The read counts of circCCDC7(15, 16, 17, 18, 19) in Chinese Prostate Cancer Genome and Epigenome Atlas (CPGEA) were calculated by Agrep as our previous study described (Wu H, et al. Cancer Lett. 2020 489:56-65). The Agrep read counts were normalized by the formula: normalized read counts=(read counts/total reads)×10⁹.

### Cell Culture

293T (ATCC, CRL-3216), HCT116 (ATCC, CCL-247EMT), PrEC LH (ATCC, PCS-440-010), LNCaP (ATCC, CRL-1740), C4-2 (ATCC, CRL-3314), PC3 (ATCC, CRL-1435), DU145 (ATCC, HTB-81), NCI-H660 (ATCC, CRL-5813), and LASCPC-01 (ATCC, CRL-3356) were originally purchased from ATCC (American Type Culture Collection) and have been confirmed by STR genotyping in our previous studies (Wang Q, et al. Cell Biosci. 2022 12(1): 153; Qin F, et al. Cancer Lett. 2017 404:53-61; Wang Q, et al. Cancer Biol Med. 2021 19(8): 1193-1210; Wang Q, et al. Front Cell Dev Biol. 2021 9:716501). We conduct routine testing for Mycoplasma every few months. PrEC LH, LNCaP, C4-2, and PC3 were cultured in RPMI 1640. 293T, HCT116 and DU145 was cultured in Dulbecco's modified Eagle's medium (Gibco, USA), supplemented with 10% fetal bovine serum (Invitrogen, USA) and 1% pen/strep (Gibco, USA). NCI-H660 and LASCPC-01 were cultured in RPMI 1640 medium, supplemented with 0.005 mg/ml Insulin, 0.01 mg/ml Transferrin, 30 nM Sodium selenite, 10 nM Hydrocortisone, 10 nM beta-estradiol, 4 mM I-glutamine (HyClone™, USA) and 10% FBS. Cells were maintained at 5% CO₂ in a 37° C. humidified incubator.

### RNA Extraction, RNase Treatment, Ribosome Extraction, and Actinomycin D Assay

Total RNA from cells was extracted using TRIzol (15596026, Thermo Fisher Scientific, USA) reagent as previously described (Wang Q, et al. Cancer Biol Med. 2021 19(8): 1193-1210). Total RNA from clinical samples was harvested according to standard procedures as our previous study did (Wang Q, et al. Front Cell Dev Biol. 2021 9:716501). In brief, 5 to 10 mg of tissue was added to a liquid nitrogen precooled mortar. The tissues were ground for 10 minutes, and 5 to 8 mL of liquid nitrogen was added every 1 min to keep the mortar cool. Then total RNA was isolated from the ground tissues by TRIzol.

cDNA was synthesized with random hexamer primer using Verso cDNA Synthesis Kit (AB-1453B, Thermo Fisher Scientific, USA). In brief, a 20 μL final reaction system was set up according to Table 1. After incubation at 42° C. for 30 minutes, cDNA was obtained, followed by incubation at 95° C. for 2 minutes to inactivate the enzyme.

TABLE 1

| | volume |
|---|---|
| 5X cDNA synthesis buffer | 4 μL |
| 5 mM dNTP Mix | 2 μL |
| 400 ng/μL Random hexamers | 1 μL |
| RT Enhancer | 1 μL |
| Verso Enzyme Mix | 1 μL |
| Template (RNA) | 1-5 μL (1000 ng) |
| Water, nuclease-free | To 20 μL |
| Total volume | 20 μL |

RNase R (RNR07250, Lucigen, USA) treatment was used on RNAs extracted from tissues or cell lines at 37° C. for 30 min followed by 65° C. 20 min to enrich for circular RNAs.

Ribosome Extraction Kit (BB3606, BestBio, China) was applied to extract ribosomes from cultured cells. In brief, 1×10⁷ cells were collected and washed two times using PBS. Ribosomes were obtained according to the kit instructions, and then RNA was extracted from ribosomes using TRIzol as previously descried (Xie Z, et al. Proc Natl Acad Sci USA. 2022 119(24): e2118048119).

The Nuclear and Cytoplasmic Extraction Kit (78833, Thermo Fisher Scientific, USA) was applied to isolate RNA from nuclear and cytoplasm fractions according to the manufacturer's instructions. The Cytosolic and Membrane Extraction Kit (89842, Thermo Fisher Scientific, USA) was applied to isolate protein from cytosolic and membrane according to the manufacturer's instructions. For actinomycin D assay, PC3 cells were treated with 2 mg/ml actinomycin D (11805017, Thermo Fisher Scientific, USA) to block transcription for 8, 16, and 24 h.

### RT-qPCR and Touch-Down PCR

Quantitative real-time PCR (qRT-PCR) was carried out on ABI StepOne Plus real time PCR system (Applied Biosystems, USA) using SYBR mix kit (AB-1285B, Thermo-Fisher Scientific, USA) as previously described (Xie Z, et al. Nat Commun. 2020 11(1): 3457). In detail, a 20 μL final reaction was prepared up according to the Table 2. All primers used in this study are listed in Table 9. The RT-qPCR reaction was performed according to the protocol in Table 3. Touch-down PCR (TD-PCR) was carried out using Platinum Taq High Fidelity Kit (11304-011, Invitrogen, USA) to amplify the full length of circCCDC7(15, 16, 17, 18, 19) using divergent primers. In detail, a 50 μL final reaction was prepared according to Table 4. The TD-PCR reaction was performed according to the protocol in Table 5.

TABLE 2

| | Volume | |
|---|---|---|
| 2X Absolute qPCR SYBR Green Capillary Mix | 10 | μL |
| 10 μM Forward primer | 0.4 | μL |
| 10 μM Reverse primer | 0.4 | μL |
| Template (cDNA) | 2 | μL |
| Water, nuclease-free | 7.2 | μL |
| Total volume | 20 | μL |

TABLE 3

| Step | Temperature (° C.) | Time | Number of cycles |
|---|---|---|---|
| Enzyme activation | 95 | 15 min | 1 cycle |
| Denaturation | 95 | 15 s | 40 cycles |
| Annealing | 60 | 30 s | |
| Extension | 72 | 45 s | |

TABLE 4

| | volume |
|---|---|
| 10× High Fidelity PCR Buffer | 5 μL |
| 50 mM MgSO₄ | 2 μL |
| 10 mM dNTP Mix | 1 μL |
| 10 μM Forward Primer | 1 μL |
| 10 μM Reverse Primer | 1 μL |
| cDNA | 2 μL (<500 ng) |
| Platinum Taq DNA Polymerase High Fidelity (5 U/μL) | 0.2 μL |
| Water, nuclease-free | To 50 μL |

TABLE 5

| | Temperature | Reaction | |
|---|---|---|---|
| Step | (° C.) | time | |
| Initial | 94° C. | 2 min | |
| denaturation | | | |
| Denature | 94° C. | 30 s | Repeat ×2/×6/×10/×26 |
| Anneal | 58° C. | 30 s | times from 58° C. to 52° C. |
| Extension | 68° C. | 12 min | |
| Final extension | 68° C. | 10 min | |
| Hole | 4° C. | indefinitely | |

### Agarose Electrophoresis and Sanger Sequencing

2% Agarose Gel was used for separating PCR products. In detail, mix agarose (17850, Thermo Fisher Scientific, USA) powder with 1×TAE (Tris-base, Acetate and EDTA solution) in a microwavable flask. Microwave for 1-3 min to completely dissolve agarose followed by adding ethidium bromide (EtBr). Pour the agarose into a gel tray with the well comb in place and wait for 20-30 mins until the gel completely solidified. PCR products were loaded into the wells of the gel and run at 120V for 30 minutes. AxyPrep DNA Gel Extraction Kit (K210025, Thermo Fisher Scientific, USA) was used for gel extraction and DNA purification and followed by Sanger sequencing at Genewiz.

### Plasmid Construction and Transient Transfection

The circCCDC7(15, 16, 17, 18, 19) and frame shift mutation fragments were cloned into the plenti-ciR-GFP-T2A vector (IGE Biotechnology, Guangzhou, China) to construct the overexpression plasmids. Before delivering the plasmids, the company conducted its own sequence validation. We have received the testing reports for all plasmids and confirmed all the sequences were correct. 293T cells were used to package virus with the helper plasmids PMD2G and psPAX2. Stable cells that overexpress circular circCCDC7(15, 16, 17, 18, 19) were selected with puromycin. The linear CCDC7 sequence was cloned into the pCDH-CMV-MCS-EF1-copGFP-T2A-Puro vector (IGE Biotechnology, Guangzhou, China) to construct the overexpression plasmid. For the transient transfection system, the plasmids above were transfected into 293T, PC3 and DU145 with X-treme GENE HP DNA Transfection Reagent (6366546001, Roche, Basel, Switzerland) and cultured for 72 h for further investigation.

RNA interference (siRNA) oligonucleotides targeting FLRT3 and negative control siRNAs were purchased from Thermo Fisher (s24376, s24377, and s24378). siRNA transfections were performed using 200 nM siRNA with 6 μL/mL Lipofectamine RNAimax (13778075, Thermo Fisher Scientific, USA) and incubated for 72 h for RNA isolation or protein collection.

### Protein Isolation and Western Blotting

Protein isolation and Western blotting were performed as described previously (Xie Z, et al. Nat Commun. 2020 11(1): 3457). Ultracel-30 regenerated cellulose membrane (UFC8030, Sigma) was used to enrich the target circCCDC7-180aa from the cell culture supernatant. Primary antibodies: FLAG [1:1000; 14793; Cell Signaling Technology (CST)], FLRT3(1:500; YN1973; ImmunoW), Slug (1:500; YN5478; ImmunoW), GAPDH (1:1000; 97166S; CST), E-cadherin (1:500; YT1454; ImmunoW) and Claudin1(1:1000; 13255; CST).

### Immunofluorescence, Immunohistochemical Scoring and ELISA

HCT116 cells were transfected with plenti-ciR circCCDC7(15, 16, 17, 18, 19)-GFP-T2A-Flag expressing plasmid for 72 h and seeded on chamber slides and subsequently co-fixed with 4% PFA and 0.5% Triton-X 100 for 10 min at 37° C. and blocked with 5% BSA in PBS. Cells were then incubated with primary antibody (FLAG) overnight at 4° C., washed with PBS, and incubated with fluorescent secondary antibody for 1 h at 37° C. Cells were then washed with PBS and incubated with DAPI for 10 min. Cells were subsequently examined under a Zeiss LSM 510 laser scanning fluorescence confocal microscope at 400× nominal magnification.

Immunohistochemical staining of FLRT3 from clinical samples were obtained from Proteinatlas. We evaluated the immunostaining intensity of each sample as follows: negative=0, weak=1, moderate=2, and strong=3. We assessed the quantity of positively stained cells: negative=0, <25%=1, 25%-75%=2 and >75%=3. The immunohistochemical score was calculated as the intensity score multiplied by the quantity score.

An epitope tag detection ELISA Kit (501560, Cayman, British Overseas) was used to detect secreted protein encoded by circCCDC7(15, 16, 17, 18, 19) in extra-cellular environment according to the manufacturer's instructions.

### Immunoprecipitation (IP) Assays and Mass Spectrometry

Co-IP assays were performed according to the manufacturer's instructions of the Pierce Crosslink Magnetic IP/Co-IP Kit (88805, Thermo Scientific) as our previous study described (Lovnicki J, et al. J Clin Invest. 2020 130(10): 5338-5348). The proteins eluted from magnetic beads were sent to Wininnovate Bio (Shenzhen, China) for mass spectrometry analysis.

### Obtaining and Concentration of the Supernatant

Three days after 293T transfected with vector or circCCDC7(15, 16, 17, 18, 19) plasmid, the supernatant was obtained and Ultra-4 Centrifugal Filter Unit (UFC803096-1, Merck millipore, USA) was used to enrich proteins in supernatant to 50 μl. The supernatant was then lyophilized by using freeze dryer (CV600, Jiaimu, Beijing).

### Cell Proliferation, Colony Formation, Migration, and Invasion Assays

For cell proliferation assay, cells (1,000 for DU145 and 1,500 for PC3 cells per well) were seeded in 96-well plates and cultured for 5 days. We measured the absorbance of each well at 450 nm every day using CCK8 (HY-K0301, MedChem Express, USA).

For the colony formation assay, cells (1,000 for DU145 and 1,500 for PC3 cells per well) were seeded in six-well plates and cultured in incubator for 7 days to form macroscopic clones. After staining with 0.1% crystal violet, we compared the difference among different groups.

The 24-well Transwell chamber (8 mM, 353097; Corning, USA) was used for the migration and invasion assay. In brief, 40,000 cells in 200 mL of 1% FBS medium were seeded in the top insert chamber, and 600 mL of medium containing 10% FBS was added into the lower chamber. The membranes in the top insert chamber were covered with Matrigel Basement Membrane Matrix (354234, Corning) for the cell invasion assay. The top chamber was fixed with 4% paraformaldehyde and stained with 0.2% crystal violet after incubation (12 h for DU145 and 48 h for PC3). The migrated cells on the lower membrane surface of the top chamber were counted under a microscope (Nikon, Tokyo, Japan).

### In Vivo Tumorigenesis Experiments

All procedures involving animals were approved by the University of Virginia Institutional Animal Care and Use Committee. Immunocompromised BALB/c adult male mice (6-8 weeks old) were used. Animals were housed in sterilized plastic cages under specific pathogen-free conditions, at 22° C., 12/12 light/dark cycle, 55% humidity. A total of 5×10⁶ DU145 cells were injected subcutaneously into both side of the dorsum (left side is control group, right side is circCCDC7(15, 16, 17, 18, 19) overexpressed group), and five mice were used here each time. At 6 weeks after implantation, the mice were euthanized by overdose Carbon dioxide (CO₂), and the tumors were surgically dissected. The isolated tumor was weighed, and the volumes of the tumor were recorded using the following formula: tumor volume (mm3)=(length [mm])×(width [mm])2×0.5.

### Statistical Analyses

Clinical quantitative paired results from CPGEA and Sun Yat-sen memorial hospital in this study were assessed by Mann-Whitney test and Wilcoxon signed rank test (SPSS 20.0, Armonk, NY, USA). All other quantitative data are presented as the mean±SD and were evaluated using GraphPad Prism 7.0. Statistical differences between the groups were assessed by one-way analysis of variance followed by Student's t-test, and p≤0.05 was considered significant. Spearman correlations were used to analyze the association of circCCDC7(15, 16, 17, 18, 19) with linear CCDC7. Fisher's exact tests were used to analyze the association of FLRT3 expression with clinicopathological characteristics by SPSS 22.0 software (SPSS Inc., Chicago, IL, USA). The Kaplan-Meier method was used to describe recurrence-free or relapse-free survival in patients from CPGEA or TCGA, and p≤0.05 was considered statistically significant after Log-rank test.

TABLE 6
Prognostic value of circCCDC7(15, 16, 17, 18, 19) expression
for the survival by Cox proportional hazards model.

| | | |
|---|---|---|
| Variable | HR (95% CI) | p |

Univariable analysis

| | | |
|---|---|---|
| Age (≥70 vs. <70) | 1.404 (0.717-2.751) | 0.323 |
| Gleason score (<7 vs. ≥7) | 0.039 (0.000-3.856) | 0.166 |
| Tumor stage (T2 vs. T3-T4) | 0.561 (0.282-1.114) | 0.099 |
| Lymph node stage (N0 vs. N1) | 0.369 (0.166-0.820) | 0.014* |
| PSA (<20 vs. ≥20) | 0.250 (0.113-0.552) | 0.001*** |
| Bone metastasis (Yes vs. No) | 0.335 (0.117-0.957) | 0.041* |
| Distant metastasis (M0 vs. M1) | 0.553 (0.192-1.597) | 0.274 |
| Seminal Vesical (Yes vs. No) | 0.338 (0.164-0.695) | 0.003** |
| Surgical Margin (Yes vs. No) | 0.540 (0.275-1.062) | 0.074 |
| circCCDC7 (Low vs. High) | 2.612 (1.070-6.378) | 0.035* |

Multivariable analysis

| | | |
|---|---|---|
| Gleason score (<7 vs. ≥7) | 0.000 (0.000-) | 0.977 |
| Lymph node stage (N0 vs. N1) | 0.500 (0.211-1.187) | 0.116 |
| PSA (<20 vs. ≥20) | 0.445 (0.186-1.063) | 0.068 |
| Bone metastasis (Yes vs. No) | 0.341 (0.106-1.091) | 0.070 |
| Seminal Vesical (Yes vs. No) | 0.433 (0.192-0.979) | 0.044* |
| circCCDC7 (Low vs. High) | 2.855 (1.132-7.354) | 0.026* |

Cut off value of circCCDC7(15, 16, 17, 18, 19) score: 10% low expression vs 90% high expression, PSA, prostate specific antigen; T, tumor; N, node; M, metastasis.
*p ≤ 0.05,
**p ≤ 0.01,
***p ≤ 0.001.
Note:
Univariable Cox regression using the log transformed circCCDC7(15, 16, 17, 18, 19) did not result in a significant result.

TABLE 7
Correlation of FLRT3 expression with clinico-pathologic
characteristics of PCa patients in TCGA database.

| | Total | | | | | |
|---|---|---|---|---|---|---|
| Clinical features | patients, n | Low, n (%) | | High, n (%) | | P |
| Age, years | | | | | | 0.745 |
| ≤65 | 354 | 175 | (49.4%) | 179 | (50.6%) | |
| >65 | 143 | 73 | (51%) | 70 | (49%) | |
| Gleason score | | | | | | 0.012* |
| ≤7 | 292 | 132 | (45.2%) | 160 | (54.8%) | |
| >7 | 205 | 116 | (56.6%) | 89 | (43.4%) | |
| Serum PSA levels, ng/ml | | | | | | 0.13 |
| ≤4 | 413 | 198 | (47.9%) | 215 | (52.1%) | |
| >4 | 27 | 17 | (63%) | 10 | (37%) | |
| T stage | | | | | | 0.364 |
| T1-T2 | 187 | 89 | (47.6%) | 98 | (52.4%) | |
| T3-T4 | 303 | 157 | (51.8%) | 146 | (48.2%) | |
| Lymph node metastasis | | | | | | 0.178 |
| N0 | 346 | 168 | (48.6%) | 178 | (51.4%) | |
| N1 | 79 | 45 | (57%) | 34 | (43%) | |
| Distant metastasis | | | | | | 0.258 |
| M0 | 454 | 229 | (50.4%) | 225 | (49.6%) | |
| M1 | 3 | 3 | (100%) | 0 | (0%) | |

Cut off value of FLRT3 score: medium; PSA, prostate-specific antigen; T, tumor; N, node; M, metastasis.
*P < 0.05.

TABLE 8
Calculation of IHC score of FLRT3 in Proteinatlas.

| Patient | Grade | intensity | Quantity | Score |
|---|---|---|---|---|
| 1 | Low | 2 | 3 | 6 |
| 2 | Low | 2 | 3 | 6 |
| 3 | Low | 2 | 2 | 4 |
| 4 | Low | 2 | 2 | 4 |
| 5 | High | 1 | 3 | 3 |
| 6 | High | 1 | 1 | 1 |
| 7 | High | 1 | 2 | 2 |
| 8 | High | 2 | 3 | 6 |
| 9 | High | 0 | 0 | 0 |
| 10 | High | 1 | 1 | 1 |
| 11 | High | 1 | 2 | 2 |
| 12 | High | 1 | 3 | 3 |
| 13 | High | 1 | 2 | 2 |
| 14 | High | 2 | 3 | 6 |
| 15 | High | 0 | 0 | 0 |
| 16 | High | 1 | 1 | 1 |
| 17 | High | 2 | 2 | 4 |
| 18 | High | 2 | 2 | 4 |

TABLE 9
The sequences of primers used in this study

| # | Target | Forward (5′-3′) | Reverse (5′-3′) |
|---|---|---|---|
| 1 | Fusion CCDC7: 19-15 | ACAAACTTACCAAGCAGCAG | AGCCTGTTTCAATTGCTCCT (SEQ ID NO: 21) |
| | | (SEQ ID NO: 20) | |
| 2 | Fusion CCDC7: 19-13 | ACAAACTTACCAAGCAGCAG | CACAGCTTTCTTTGTTCGAT (SEQ ID NO: 23) |
| | | (SEQ ID NO: 22) | |
| 3 | Linear CCDC7 | TCCgTACAACgTCAAgAAgg | AAAgCTTggACCAgCCCTAT (SEQ ID NO: 25) |
| | | (SEQ ID NO: 24) | |
| 4 | Divergent CCDC7: 19-15 | AATCTCAAGAAAGTAGCACGTC | CTGCTGCTTGGTAAGTTTGTT (SEQ ID NO: 27) |
| | | (SEQ ID NO: 26) | |
| 5 | All isoforms of | CAAGATTCACTGTCTGTTGG | AGCCTGTTTCAATTGCTCCT (SEQ ID NO: 29) |
| | CircCCDC7 | (SEQ ID NO: 28) | |
| | (15, 16, 17, 18, 19) | | |
| 6 | Specific isoform of | AACGATCTAGCCCTGCTATT | AGCCTGTTTCAATTGCTCCT (SEQ ID NO: 31) |
| | CircCCDC7 | (SEQ ID NO: 30) | |
| | (15, 16, 17, 18, 19) | | |
| 7 | circFBWX7 | CAGTGTCACGAACTCCAGTA | CACATTCCTCTGACCCAGTA (SEQ ID NO: 33) |
| | | (SEQ ID NO: 32) | |
| 8 | CircITCH | AAGGAGCAATGCAGCAGTTT | AGTCACAACTACTTCTTCAACCCAT (SEQ ID |
| | | (SEQ ID NO: 34) | NO: 35) |
| 9 | FLRT3 | ACAGTGTATCCTGCCCAAGG | AGCCAGCTGAGTCTCAAAGC (SEQ ID NO: 37) |
| | | (SEQ ID NO: 36) | |
| 10 | GAPDH | CAAGGCTGAGAACGGGAAG | TGAAGACGCCAGTGGACTC (SEQ ID NO: 39) |
| | | (SEQ ID NO: 38) | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A synthetic linear polynucleotide comprising the nucleic acid sequence SEQ ID NO:2, or a variant thereof having at least 95% sequence identity to SEQ ID NO:2, a fragment thereof encoding at least amino acids 174-180 of SEQ ID NO:1, or a combination thereof, wherein the linear polynucleotide comprises a cap and poly (A) tail.

2. The synthetic oligonucleotide of claim 1, wherein the synthetic oligonucleotide has one or modified nucleosides, at least one modified internucleoside linkage, or a combination thereof.

3. The synthetic oligonucleotide of claim 2, wherein the one or more modified nucleosides is a 2′ sugar modified nucleoside.

4. The synthetic oligonucleotide of claim 3, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

5. The synthetic oligonucleotide of claim 2, wherein the internucleoside linkages are phosphorothioate internucleoside linkages.

6. A peptidomimetic comprising the amino acid sequence SEQ ID NO:1, or a variant thereof having at least 95% sequence identity to SEQ ID NO:1, a fragment thereof having at least amino acids 174-180 of SEQ ID NO:1, or a combination thereof.

7. The peptidomimetic of claim 6, wherein the peptidomimetic is a fragment having at least 10, 20, 30, 40, 50, 60, 70, 80, 90, 100, 110, 120, 130, 140, 150, 160, 170, 180 contiguous amino acids from SEQ ID NO:1.

8. The peptidomimetic of claim 6, wherein the peptidomimetic further comprises a signal peptide, a C-terminal tag, or a combination thereof.

9. The peptidomimetic of claim 6, wherein the peptidomimetic comprises the amino acid
sequence
(SEQ ID NO: 3)
KMTEEQTYQAAEKSQESSTSGN,
(SEQ ID NO: 4)
MTEEQTYQAAEKSQESSTSGN,
(SEQ ID NO: 5)
TEEQTYQAAEKSQESSTSGN,
(SEQ ID NO: 6)
EEQTYQAAEKSQESSTSGN,
(SEQ ID NO: 7)
EQTYQAAEKSQESSTSGN,
(SEQ ID NO: 8)
QTYQAAEKSQESSTSGN,
(SEQ ID NO: 9)
TYQAAEKSQESSTSGN,
(SEQ ID NO: 10)
YQAAEKSQESSTSGN,
(SEQ ID NO: 11)
QAAEKSQESSTSGN,
(SEQ ID NO: 12)
AAEKSQESSTSGN,
(SEQ ID NO: 13)
AEKSQESSTSGN,
(SEQ ID NO: 14)
EKSQESSTSGN,
(SEQ ID NO: 15)
KSQESSTSGN,
(SEQ ID NO: 16)
SQESSTSGN,
(SEQ ID NO: 17)
QESSTSGN,
or
(SEQ ID NO: 18)
ESSTSGN.

10. A chimeric polypeptide comprising the amino acid sequence SEQ ID NO:1, or a variant thereof having at least 95% sequence identity to SEQ ID NO:1, a fragment thereof having at least amino acids 174-180 of SEQ ID NO:1, or a combination thereof, fused to a signal peptide.

11. The chimeric polypeptide of claim 10, wherein the fragment of SEQ ID NO:1 comprises at least 10, 20, 30, 40, 50, 60, 70, 80, 90, 100, 110, 120, 130, 140, 150, 160, 170, 180 contiguous amino acids from SEQ ID NO:1.

12. The chimeric polypeptide of claim 10, further comprising a C-terminal tag.

13. The chimeric polypeptide of claim 10, wherein the fragment of SEQ ID NO:1 consists of the amino acid sequence KMTEEQTYQAAEKSQESSTSGN (SEQ ID NO:3), MTEEQTYQAAEKSQESSTSGN (SEQ ID NO:4), TEEQTYQAAEKSQESSTSGN (SEQ ID NO: 5), EEQTYQAAEKSQESSTSGN (SEQ ID NO:6), EQTYQAAEKSQESSTSGN (SEQ ID NO: 7), QTYQAAEKSQESSTSGN (SEQ ID NO:8), TYQAAEKSQESSTSGN (SEQ ID NO:9), YQAAEKSQESSTSGN (SEQ ID NO:10), QAAEKSQESSTSGN (SEQ ID NO:11), AAEKSQESSTSGN (SEQ ID NO:12), AEKSQESSTSGN (SEQ ID NO:13), EKSQESSTSGN (SEQ ID NO:14), KSQESSTSGN (SEQ ID NO:15), SQESSTSGN (SEQ ID NO:16), QESSTSGN (SEQ ID NO:17), or ESSTSGN (SEQ ID NO:18).

14. A method for treating an FLRT3-attenuated cancer in a subject, the method comprising administering to the subject a composition comprising:
(a)(i) a DNA expression vector expressing an mRNA polynucleotide comprising the nucleic acid sequence SEQ ID NO:2, or a variant thereof having at least 95% sequence identity to SEQ ID NO:2, a fragment thereof encoding at least amino acids 174-180 of SEQ ID NO:2; or
a combination thereof, wherein the linear polynucleotide comprises a cap and poly (A) tail, or (a)(ii) a synthetic oligonucleotide comprising the nucleic acid sequence SEQ ID NO:2, or a variant thereof having at least 95% sequence identity to SEQ ID NO:2, a fragment thereof encoding at least amino acids 174-180 of SEQ ID NO:2; or a combination thereof, wherein the linear polynucleotide comprises a cap and poly (A) tail, or
(a)(iii) a polypeptide or peptidomimetic having the amino acid sequence SEQ ID NO:1, or a variant thereof having at least 95% sequence identity to SEQ ID NO:1, a fragment thereof having at least amino acids 174-180 of SEQ ID NO:1, or a combination thereof; and
(b) a pharmaceutically acceptable excipient.

15. The method of claim 14, wherein the FLRT3-attenuated cancer is a prostate cancer, pancreatic cancer, lung cancer, renal clear cell cancer, or colon cancer.

16. The method of claim 14, wherein the polypeptide or peptidomimetic is a fragment having at least 10, 20, 30, 40, 50, 60, 70, 80, 90, 100, 110, 120, 130, 140, 150, 160, 170, 180 contiguous amino acids from SEQ ID NO:1.

17. The method of claim 14, wherein the synthetic oligonucleotide is a linear oligonucleotide comprising a cap and poly (A) tail.

18. The method of claim 17, wherein the synthetic oligonucleotide comprises one or modified nucleosides, at least one modified internucleoside linkage, or a combination thereof.

19. The method of claim 18, wherein the one or more modified nucleosides is a 2′ sugar modified nucleoside.

20. The method of claim 18, wherein the internucleoside linkages are phosphorothioate internucleoside linkages.


SEQUENCE LISTING (39 sequences)

SEQ ID NO: 1 - protein, 180 aa, unidentified
MQVGNSQTKVKGEDSKNIPLEKETRKSLVSDSGGQRTSDKIQEYPQITAQSGRLIEKSSEKKRSSPAISDLSQILKSQDESAFLESSNEVSVAENQSYKSPSETHDKSLTTVSSSKEVQDSLSVGTLAQKNETVISPFILPPVLTESKKADVSEEQLQKMTEEQTYQAAEKSQESSTSGN

SEQ ID NO: 2 - RNA, 543 nt, unidentified
  source 1..543: mol_type = mRNA
atgcaagtgggtaatagtcaaacaaaagttaaaggtgaagattcaaaaaatataccattggagaaagaaacaagaaaatcactggtttcagattcaggtggacaaaggacaagtgataaaatccaagaatatccacagatcactgcccaaagcggaagactgattgaaaagagctcagagaagaaacgatctagccctgctatttcagatctttcacagatccttaagtctcaagatgaatcagcatttttagagagttcaaatgaagtttcagttgctgaaaaccaatcctataaatctccatcagagacccatgataaatcacttacaacagtatcatccagcaaagaagttcaagattcactgtctgttggaacattggctcaaaaaaacgaaacagtgatatcaccattcattttacctcctgttcttacagaaagtaaaaaggctgatgtttcagaagaacaattacaaaagatgactgaagaacaaacttaccaagcagcagaaaaatctcaagaaagtagcacgtctggaaattga

SEQ ID NO: 3 - protein, 22 aa, unidentified
KMTEEQTYQAAEKSQESSTSGN

SEQ ID NO: 4 - protein, 21 aa, unidentified
MTEEQTYQAAEKSQESSTSGN

SEQ ID NO: 5 - protein, 20 aa, unidentified
TEEQTYQAAEKSQESSTSGN

SEQ ID NO: 6 - protein, 19 aa, unidentified
EEQTYQAAEKSQESSTSGN

SEQ ID NO: 7 - protein, 18 aa, unidentified
EQTYQAAEKSQESSTSGN

SEQ ID NO: 8 - protein, 17 aa, unidentified
QTYQAAEKSQESSTSGN

SEQ ID NO: 9 - protein, 16 aa, unidentified
TYQAAEKSQESSTSGN

SEQ ID NO: 10 - protein, 15 aa, unidentified
YQAAEKSQESSTSGN

SEQ ID NO: 11 - protein, 14 aa, unidentified
QAAEKSQESSTSGN

SEQ ID NO: 12 - protein, 13 aa, unidentified
AAEKSQESSTSGN

SEQ ID NO: 13 - protein, 12 aa, unidentified
AEKSQESSTSGN

SEQ ID NO: 14 - protein, 11 aa, unidentified
EKSQESSTSGN

SEQ ID NO: 15 - protein, 10 aa, unidentified
KSQESSTSGN

SEQ ID NO: 16 - protein, 9 aa, unidentified
SQESSTSGN

SEQ ID NO: 17 - protein, 8 aa, unidentified
QESSTSGN

SEQ ID NO: 18 - protein, 7 aa, unidentified
ESSTSGN

SEQ ID NO: 19 - protein, 9 aa, unidentified
SQESSTSGN

SEQ ID NO: 20 - DNA, 20 bp, unidentified
  source 1..20: mol_type = other DNA
acaaacttaccaagcagcag

SEQ ID NO: 21 - DNA, 20 bp, unidentified
  source 1..20: mol_type = other DNA
agcctgtttcaattgctcct

SEQ ID NO: 22 - DNA, 20 bp, unidentified
  source 1..20: mol_type = other DNA
acaaacttaccaagcagcag

SEQ ID NO: 23 - DNA, 20 bp, unidentified
  source 1..20: mol_type = other DNA
cacagctttctttgttcgat

SEQ ID NO: 24 - DNA, 20 bp, unidentified
  source 1..20: mol_type = other DNA
tccgtacaacgtcaagaagg

SEQ ID NO: 25 - DNA, 20 bp, unidentified
  source 1..20: mol_type = other DNA
aaagcttggaccagccctat

SEQ ID NO: 26 - DNA, 22 bp, unidentified
  source 1..22: mol_type = other DNA
aatctcaagaaagtagcacgtc

SEQ ID NO: 27 - DNA, 21 bp, unidentified
  source 1..21: mol_type = other DNA
ctgctgcttggtaagtttgtt

SEQ ID NO: 28 - DNA, 20 bp, unidentified
  source 1..20: mol_type = other DNA
caagattcactgtctgttgg

SEQ ID NO: 29 - DNA, 20 bp, unidentified
  source 1..20: mol_type = other DNA
agcctgtttcaattgctcct

SEQ ID NO: 30 - DNA, 20 bp, unidentified
  source 1..20: mol_type = other DNA
aacgatctagccctgctatt

SEQ ID NO: 31 - DNA, 20 bp, unidentified
  source 1..20: mol_type = other DNA
agcctgtttcaattgctcct

SEQ ID NO: 32 - DNA, 20 bp, unidentified
  source 1..20: mol_type = other DNA
cagtgtcacgaactccagta

SEQ ID NO: 33 - DNA, 20 bp, unidentified
  source 1..20: mol_type = other DNA
cacattcctctgacccagta

SEQ ID NO: 34 - DNA, 20 bp, unidentified
  source 1..20: mol_type = other DNA
aaggagcaatgcagcagttt

SEQ ID NO: 35 - DNA, 25 bp, unidentified
  source 1..25: mol_type = other DNA
agtcacaactacttcttcaacccat

SEQ ID NO: 36 - DNA, 20 bp, unidentified
  source 1..20: mol_type = other DNA
acagtgtatcctgcccaagg

SEQ ID NO: 37 - DNA, 20 bp, unidentified
  source 1..20: mol_type = other DNA
agccagctgagtctcaaagc

SEQ ID NO: 38 - DNA, 19 bp, unidentified
  source 1..19: mol_type = other DNA
caaggctgagaacgggaag

SEQ ID NO: 39 - DNA, 19 bp, unidentified
  source 1..19: mol_type = other DNA
tgaagacgccagtggactc